(19) United States
(12) Patent Application Publication
(10) Pub. No.: US 2017/0100385 A1
(43) Pub. Date: Apr. 13, 2017

(54) PHARMACEUTICAL COMPOSITIONS COMPRISING DANIRIXIN FOR TREATING INFECTIOUS DISEASES

(71) Applicant: GlaxoSmithKline Intellectual Property (No. 2) Limited, Brentford, Middlesex (GB)

(72) Inventor: Michael WASHBURN, Research Triangle Park, NC (US)

(73) Assignee: GLAXOSMITHKLINE INTELLECTUAL PROPERTY (NO. 2) LIMITED, Brentford, Middlesex (GB)

(21) Appl. No.: 15/310,592

(22) PCT Filed: May 8, 2015
(86) PCT No.: PCT/IB2015/053373

§ 371 Date: Nov. 11, 2016

Related U.S. Application Data
(60) Provisional application No. 62/151,013, filed on Apr. 22, 2015.
(60) Provisional application No. 62/149,893, filed on Apr. 20, 2015.
(60) Provisional application No. 61/991,754, filed on May 12, 2014.

(51) Int. Cl. A61K 31/4462; A61K 31/215; A61K 31/7056; C12N 15/113; A61K 31/5377; A61K 31/4545; A61K 31/55; A61K 31/437; A61K 31/422; A61K 31/41; A61K 31/433; A61K 31/7068; A61K 31/519; A61K 31/4184; A61K 31/4196; C07K 16/10; A61K 39/42; A61K 9/00; A61K 47/40; A61K 9/08; A61K 31/5513; A61K 31/351

(52) U.S. Cl. CPC A61K 31/4462; A61K 31/351; A61K 31/215; A61K 31/7056; C12N 15/1131; A61K 31/5377; A61K 31/4545; A61K 31/55; A61K 31/437; A61K 31/422; A61K 31/41; A61K 31/433; A61K 31/7068; A61K 31/519; A61K 31/4184; A61K 31/4196; C07K 16/1027; A61K 39/42; A61K 9/0019; A61K 47/40; A61K 9/08; A61K 31/5513; C07K 2317/24; C12N 2310/14; C12N 2320/31; C12N 2310/11

(57) ABSTRACT

Provided are compounds and pharmaceutically acceptable salts thereof, and combinations of compounds, their pharmaceutical compositions, their methods of preparation, and methods for their use in treating or preventing infectious disease.

### CROSS REFERENCE TO RELATED PATENTS AND PATENT APPLICATIONS

This is a Patent Cooperation Treaty Application and claims the benefit of U.S. Provisional Patent Application No. 61/991,754, filed on May 12, 2014; U.S. Provisional Patent Application No. 62/149,893, filed on Apr. 20, 2015; and U.S. Provisional Patent Application No. 62/151,013, filed on Apr. 22, 2015; which are all hereby incorporated by reference in their entirety.

### FIELD OF THE INVENTION

The present invention relates to certain compounds, methods and pharmaceutical compositions for treating infectious diseases, such as viral and bacterial infections. Methods for preparing such compounds and methods of using the compounds are also disclosed. In particular, the treatment of viral infections such as those caused by Paramyxoviridae, Orthomyxoviridae, Flaviviridae, Picornaviridae, and Coronaviridae are disclosed.

### BACKGROUND OF THE INVENTION

CXCR2 is a chemokine receptor that is highly expressed on neutrophils, and signaling through this receptor causes inflammatory cell recruitment to the injured tissue (1-2). For example, it has been noted that RSV-infected infants have increased neutrophils in the lungs (3-6). In addition, genetic single nucleotide polymorphisms (SNP's) that increase production of the CXCR2 ligand, IL-8, are associated with RSV bronchiolitis and wheezing (7,8). Neutrophils are also a prominent cell type that is recruited to the lung during influenza infection, and ablation of CXCR2 during influenza infection in mice significantly reduced neutrophil infiltration into the lung (9,10).

Mucus overproduction during RSV infection is known to be detrimental to infants because it blocks the small airways of the lungs and prevents proper oxygen exchange. In a mouse model of RSV infection, signaling via CXCR2 contributes to mucus overproduction and airway hyperresponsiveness. Immunoneutralization with an anti-CXCR2 antibody and CXCR2 ⁺mice showed a significant reduction of mucus in the lungs after RSV infection (11). It was also reported that influenza infected mice treated with a CXCR2 ligand antibody (MIP-2), demonstrated reduced lung neutrophil counts along with an improvement in lung pathology without affecting viral replication and clearance (12). In summary, CXCR2 and some of its ligands (e.g., IL-8), have been shown to be significantly upregulated during respiratory infections in humans.

Therefore, compounds which are capable of binding to the CXCR2 receptor and inhibit CXCR2 ligand (e.g., IL-8) binding could help treat conditions associated with an increase in CXCR2 ligand production. Such compounds could, therefore, treat inflammatory conditions associated with CXCR2 ligand induced chemotaxis of neutrophils. Acute viral and bacterial lung infections cause significant immune inflammation and mucus production, which often leads to clogged airways, difficulty breathing, and hospitalization. Current antiviral treatments and antibiotics work with varying degrees of success when administered shortly after symptom onset. While the infectious agent plays a role in disease and pathogenesis, the overzealous immune response to the infection also significantly contributes to the etiology of severe respiratory illnesses.

Influenza viruses are a global health concern, having been responsible for three major pandemics that have killed over fifty million people worldwide since the year 1900. Recently, The World Health Organization has estimated that there are three to five million cases of severe influenza each year, and as many as five hundred thousand of these individuals die annually from complications. See WHO, Fact sheet N° 211, (2009). Influenza is characterized by a sudden onset of high fever, cough, headache, muscle and joint pain, severe malaise, sore throat and runny nose. These symptoms are believed to be the result of an over or unspecific reaction of the immune system. Most people recover from fever and other symptoms within a week without requiring medical attention. However, influenza can cause severe illness or death in people at high risk. Id. Indeed, the highest risk of complications occur among children younger than age two, adults age 65 or older, and people of any age with certain medical conditions, such as chronic heart, lung (La, COPD and asthma), kidney, liver, blood or metabolic diseases (i.e., diabetes), or those with weakened immune systems.

Current therapeutic agents against infections with various influenza viruses focus on disrupting the action of neuraminidase. Before the transmission of the influenza viruses to other cells can occur, the sialic acid on the cell surface needs to be cleaved with the viral protein neuraminidase. Tamiflu® (oseltamivir phosphate) is a neuraminidase inhibitor that is administered orally, and Relenza® (zanamivir) is a neuraminidase inhibitor that is inhaled by mouth. Other approved therapeutics, like amantadine and rimantadine, target the viral ion channel (M2 protein) and inhibit virus uncoating. Unfortunately, Tamiflu® has been reported to have serious side effects, including nausea, vomiting, and abnormalities of the nervous or mental system. Also, outbreaks of Tamiflu® -resistant viruses and amantadine-resistant viruses have been reported, including the occurrence of human-to-human transmission of resistant virus. In fact, the U.S. CDC has recommended that amandatine and rimantadine no longer be prescribed to treat influenza since such a high percentage of recent seasonal strains have shown resistance to its action. Another drawback is that many of these therapeutics are much less effective if treatment is not started within forty-eight hours of the onset of symptoms. While vaccines against certain strains of influenza can be taken prophylactically, the U.S. CDC and vaccine manufacturers must accurately predict the specific strains that will be spread in the upcoming season, a prediction that can be difficult to make.

As such, additional medical therapies are needed which could be beneficial that target multiple aspects of a respiratory infection, including, for example, mucus overproduction, airway hyperresponsiveness, and that could also inhibit replication of the underlying infectious agent.

### SUMMARY OF THE INVENTION

In accordance with one embodiment of the present invention, there is provided a novel method of treating a respiratory infection in a subject suffering from the respiratory infection comprising administering to the subject the compound of Formula (I),

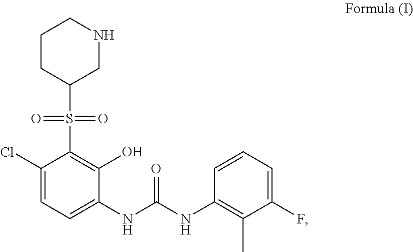 [SMILES: Cc1c(F)cccc1NC(=O)Nc1ccc(Cl)c(S(=O)(=O)C2CCCNC2)c1O]

or a pharmaceutically acceptable salt thereof, alone or in combination with an antimicrobial agent, or a pharmaceutically acceptable salt thereof. Such “combinations” of the compound of Formula (I) and an antimicrobial agent, such as, for example, any neuraminidase inhibitor, can be administered to a subject suffering from a respiratory infection as a fixed dose combination in the same dose, or such combinations can be administered in multiple separate doses.

Also provided is a composition comprising the compound of Formula (I):

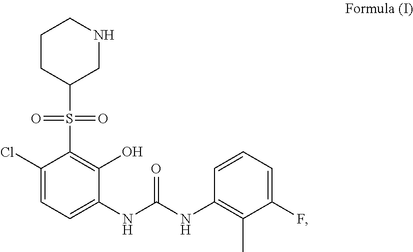 [SMILES: Cc1c(F)cccc1NC(=O)Nc1ccc(Cl)c(S(=O)(=O)C2CCCNC2)c1O]

in combination with a neuraminidase inhibitor compound.\

Also provided is a composition comprising the compound of Formula (I):

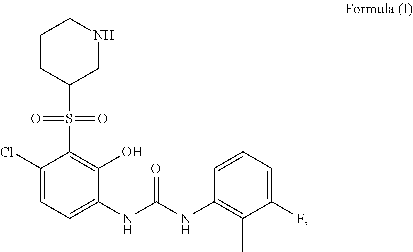 [SMILES: Cc1c(F)cccc1NC(=O)Nc1ccc(Cl)c(S(=O)(=O)C2CCCNC2)c1O]

in combination with ribavirin.

Also provided are pharmaceutical compositions comprising a pharmaceutically acceptable carrier or excipient and the compound of Formula (I), or a pharmaceutically acceptable salt thereof, in combination with an antimicrobial agent, or a pharmaceutically acceptable salt thereof.

Also provided are methods of preventing a respiratory infection in a subject comprising administering to a subject at risk of, or predisposed to, acquiring a respiratory infection, the compound of Formula (I), or a pharmaceutically acceptable salt thereof, alone or in combination with antimicrobial agent, or a pharmaceutically acceptable salt thereof.

Also provided are pharmaceutical compositions comprising a pharmaceutically acceptable carrier or excipient and the compound of Formula (I), or a pharmaceutically acceptable salt thereof, alone or in combination with an antimicrobial agent, or a pharmaceutically acceptable salt thereof.

Also provided are methods for preparing combinations of the compound of Formula (I), or a pharmaceutically acceptable salt, and an antimicrobial agent, and compositions thereof and for therapeutic uses of the combination.

### DETAILED DESCRIPTION OF REPRESENTATIVE EMBODIMENTS

Throughout this application, references are made to various embodiments relating to compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

It is to be understood that the terminology used herein is for the purpose of describing particular embodiments only and is not intended to limit the scope of the present invention. In this specification and in the claims that follow, reference will be made to a number of terms that shall be defined to have the following meanings.

An “antimicrobial agent(s)”, as used herein, refers to an agent, either a chemical compound or biological entity that kills microorganisms or inhibits their growth or prevents or counteracts their pathogenic action. Antimicrobial agents can be grouped according to the microorganisms they act primarily against, such as antivirals or antibacterials.

“Compound”, “compounds”, “chemical”, and “chemical compounds” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

“Racemates” refers to a mixture of enantiomers. In an embodiment of the invention, the the compound of Formula (I), or pharmaceutically acceptable salts thereof, are enantiomerically enriched with one enantiomer wherein all of the chiral carbons referred to are in one configuration. In general, reference to an enantiomerically enriched compound or salt, is meant to indicate that the specified enantiomer will comprise more than 50% by weight of the total weight of all enantiomers of the compound or salt.

“Solvate” or “solvates” of a compound refer to those compounds, as defined above, which are bound to a stoichiometric or non-stoichiometric amount of a solvent. Solvates of a compound includes solvates of all forms of the compound. In certain embodiments, solvents are volatile, non-toxic, and/or acceptable for administration to humans in trace amounts. Suitable solvates include water wherein the solvate is a hydrate.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

In one embodiment, the pharmaceutically acceptable salt is a hydrobromide salt of the compound of Formula (I).

“Patient” or “subject” refers to mammals and includes humans and non-human mammals.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

In accordance with one embodiment of the present invention, there is provided a medical therapy and treatment for infectious diseases of the respiratory system. In one embodiment, the present invention is useful for the treatment of symptoms caused by an infection with viruses including, but not limited to, influenza virus, human rhinovirus, other enterovirus, respiratory syncytial virus, parainfluenza virus, metapneumovirus, coronavirus, herpesviruses, or adenovirus. It should also be noted that the respiratory viral infection treated herein may also be associated with a subsequent secondary bacterial infection.

CXCR2 is a chemokine receptor that is highly expressed on neutrophils, and signaling through this receptor causes inflammatory cell recruitment to the injured tissue. Chemical antagonism of cytokine signaling to reduce neutrophil chemotaxis is expected to benefit a subject suffering from a respiratory infection by controlling, reducing, and alleviating many of the resultant symptoms by decreasing the infiltration of neutrophils. As a result, the present invention provides a novel treatment comprising the compound of Formula (I) that antagonizes the CXCR2 receptor, alone or in combination with an antimicrobial agent. For example, the inventions are expected to reduce pathogen titers and prevent repeated inflammatory cell signaling and infiltration into the lung of infected patients, which could alleviate disease symptoms and lung pathology. The present invention also provides therapeutic compositions and methods to reduce both the excessive inflammatory immune response and the replication of the virus or bacteria.

In one embodiment, the combination treatment of a CXCR2 antagonist compound (e.g., the compound of Formula I) with an antimicrobial agent is expected to target both viral/bacterial and immune aspects of disease, thereby accelerating recovery and resolution of disease, potentially faster than either treatment alone.

In other embodiments, additional agents could be added to the therapy of the CXCR2 antagonist compound of Formula I in combination with the antimicrobial. Such additional agents could comprise any other respiratory infection therapies which are efficacious to reduce one or more symptoms, including, for example, high fever, cough, headache, muscle and joint pain, malaise, sore throat, and runny nose.

The compound of Formula I is also useful in combination with antimicrobial agents for treating symptoms of an infection in a human caused by bacteria, in particular respiratory infections. Specific bacteria include, but are not limited to, the causative agents of bacterial pneumonia such as Streptococcus pneumoniae, Staphylococcus aureus, Haemophilus influenza, Klebsiella pneumoniae, Legionella pneumophila, Porphyromonas gingivalis, and Acinetobacter baumanii. In addition, the present invention is directed to respiratory infections which exacerbate underlying chronic conditions such as asthma, chronic bronchitis, chronic obstructive pulmonary disease, otitis media and sinusitis. In such a case, an infection may act as the trigger for exacerbation, and control of symptoms with the present invention would reduce the likelihood of an exacerbation occurring.

In accordance with the present invention, it has been discovered that infectious diseases and infectious disease-related complications may be treated and prevented in a subject by administering to the subject the compound of Formula (I) alone or in combination with one or more antimicrobial agents. For purposes of the present invention, the novel combination therapy comprising the compound of Formula (I) in combination with at least one antimicrobial agent is also useful for the purpose of preventing and treating infectious diseases and infectious disease-related complications in a subject that is in need of such prevention or treatment. Thus, the combination therapy of the present invention would be useful, for example, to reduce such infectious disease symptoms as, for example, coughing, rhinorrhea, breathing difficulty, shortness of breath, pain, inflammation, itchy and/or watery eyes, nasal discharge, nasal congestion, facial pressure, sneezing, sore throat, cough, headache, fever, malaise, fatigue, weakness, and/or muscle pain, in a subject suffering from such symptoms. The combination therapy of the present invention would also be useful to prevent the occurrence of such symptoms.

The methods and compositions of the present invention are also useful to reduce the number of hospitalizations of subjects suffering from an infectious disease, or to prevent or retard, in subjects, the development of complications associated with infectious diseases, which may eventually arise from having a chronic or recurring infectious disease. The combination therapy of the compound of Formula (I) and an antimicrobial agent is also useful for decreasing the required number of separate dosages, thus, potentially improving patient compliance. The administration of the compound of Formula (I) for the prevention and treatment of infectious diseases and infectious disease-related complications is an unexpectedly effective treatment and preventative therapy. Such administration is effective for improving the symptoms of infectious diseases and infectious disease-related complications while avoiding or reducing certain disadvantages of current treatments. Furthermore, the administration of the compound of Formula (I) in combination with an antimicrobial agent is an effective treatment for infectious diseases or infectious disease-related complications or symptoms, and in some embodiments, may be superior to the use of either agent alone. For example, the combination therapy could be effective for lowering the dosages of antimicrobial agents that are normally prescribed as a monotherapy. The administration of lower dosages of conventional treatment agents could provide a reduction in side effects corresponding to such conventional agents. Combination therapies comprising the compound of Formula (I) and an antimicrobial agent could be useful not only for improving infectious disease symptoms and shortening recovery times, but perhaps also for reducing the dosages of antimicrobial agents that are normally required.

As used herein, the phrases “combination therapy”, “co-administration”, “co-administering”, “administration with”, “administering”, “combination”, or “co-therapy”, when referring to use of the compound of Formula (I) in combination with an antimicrobial agent, are intended to embrace administration of each agent in a sequential manner in a regimen that will provide beneficial effects of the drug combination, and is intended as well to embrace co-administration of these agents in a substantially simultaneous manner. Thus, the compound of Formula (I) and antimicrobial agent may be administered in one therapeutic dosage form, such as in a single capsule, tablet, injection or infusion, or in two separate therapeutic dosage forms, such as in separate capsules, tablets, injections, or infusions. In other embodiments, where the compound of Formula (I) is administered in a separate dosage form relative to the antimicrobial agent, such separate dosing may be performed over similar or different time frames depending upon the therapeutic needs in a patient. One of skill in the art will understand how to appropriately time such separate dosing periods.

Sequential administration of such treatments encompasses both relatively short and relatively long periods between the administration of each of the drugs of the present method. However, in some embodiments of the present invention, the second drug is administered while the first drug is still having an efficacious effect on the subject. Thus, the present invention, in one embodiment, takes advantage of the fact that the simultaneous presence of the combination of the compound of Formula (I) and an antimicrobial agent in a subject has a greater clinical efficacy than the administration of either agent alone. Alternatively, in some embodiments of the present invention, the second drug is administered while the first drug has stopped having an efficacious effect on the subject.

In one embodiment, the second of the two drugs is to be given to the subject within the therapeutic response time of the first drug to be administered. For example, the present invention encompasses administration of the compound of Formula (I) to the subject and the later administration of an antimicrobial agent, as long as the antimicrobial agent is administered to the subject while the compound of Formula (I) is still present in the subject at a level, which in combination with the level of the antimicrobial agent is therapeutically effective, and vice versa.

As used herein, the terms “therapeutic response time” mean the duration of time that a compound is present or detectable within a subject's body at therapeutic concentrations.

As used herein, the term “monotherapy” is intended to embrace administration of the compound of Formula (I) to a subject suffering from an infectious disease or infectious disease-related complication as a single therapeutic treatment without an additional therapeutic treatment comprising an antimicrobial agent. However, the compound of Formula (I) may still be administered in multiple dosage forms. Thus, the compound of Formula (I) may be administered in one therapeutic dosage form, such as in a single capsule, tablet, injection or infusion, or in two separate therapeutic dosage forms, such as in separate capsules, tablets, injections, or infusions.

The amounts of the compound of Formula (I), or salts thereof, and the other pharmaceutically active agent(s) described herein and the relative timings of administration will be selected in order to achieve the desired combined therapeutic effect.

In other embodiments, the compounds of the present invention may be used in combination with one or more antimicrobial agents useful in the prevention or treatment of viral diseases or associated pathophysiology. Thus, the compounds of the present invention and their salts, solvates, or other pharmaceutically acceptable derivatives thereof, may be employed alone or in combination with other antimicrobial agents. The compounds of the present invention and any other pharmaceutically active agent(s) may be administered together or separately and, when administered separately, administration may occur simultaneously or sequentially, in any order. The amounts of the compounds of the present invention and the other pharmaceutically active agent(s) and the relative timings of administration will be selected in order to achieve the desired combined therapeutic effect. The administration in combination of a compound of the present invention and salts, solvates, or other pharmaceutically acceptable derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

In one embodiment, the present invention encompasses a method for preventing an infectious disease in a subject, the method comprising administering to the subject the compound of Formula (I) alone or in combination with an antimicrobial agent.

As used herein, the terms “toprevent”, “preventing”, or “prevention” refer to any reduction, no matter how slight, of a subject's predisposition or risk for developing an infectious disease or an infectious disease-related complication. For purposes of prevention, the subject is any subject, and preferably is a subject that is at risk for, or is predisposed to, developing an infectious disease or an infectious disease-related complication. The term “prevention” includes either preventing the onset of a clinically evident infectious disease altogether or preventing the onset of a preclinically evident infectious disease in individuals at risk.

In another embodiment, the present invention encompasses a method for treating an infectious disease or an infectious disease-related complication in a subject, the method comprising administering to the subject the compound of Formula (I) alone or in combination with an antimicrobial agent.

As used herein, the terms “treating”, “treatment”, “treated”, or “to treat,” mean to alleviate symptoms, eliminate the causation either on a temporary or permanent basis, or to alter or slow the appearance of symptoms or symptom worsening. These terms also include alleviation or elimination of causation of symptoms associated with, but not limited to, any of the infectious diseases or infectious disease related-complications described herein. Such terms also include reducing the duration of an infectious disease or infectious disease related-complication in a subject.

Without being bound by this or any other theory, it is believed that a therapy comprising the compound of Formula (I) is efficacious for impairing processes of inflammation within the lungs during a respiratory infection, thus preventing or treating infectious disease symptoms and thereby infectious disease-related complications. Moreover, in preferred embodiments, the combination of the compound of Formula (I) and an antimicrobial agent may provide synergistic effects, which would reduce the symptoms associated with infectious diseases and infectious disease-related complications to a greater extent than would be expected on the basis of the use of either one alone.

The term “synergistic” refers to the combination of the compound of Formula (I) and an antimicrobial agent as a combined therapy having an efficacy for the prevention and treatment of infectious diseases that could be greater than the sum of their individual effects. The synergistic effects of certain embodiments of the present invention's combination therapy could encompass additional unexpected advantages for the treatment and prevention of infectious diseases. Such additional advantages could include, but are not limited to, lowering the required dose of antimicrobial agents, reducing the side-effects of antimicrobial agents, and rendering those agents more tolerable to subjects undergoing infectious disease therapy.

Also, the monotherapy and combination therapy of the present invention could provide for the treatment or prevention of infectious disease-related complications, which may arise indirectly from having a respiratory infectious disease, by treating the underlying respiratory infectious disease itself. For example, if a subject is suffering from a viral respiratory disease-related complication, such as a secondary respiratory bacterial infection (e.g., pneumonia), the treatment of the underlying viral infectious disease, such as viral influenza, by the methods and compositions of the present invention can prevent the occurrence of the associated bacterial infection complication and it's symptoms. The present invention is directed to a novel method of preventing or treating infectious diseases and infectious disease-related complications in a subject that is in need of such prevention or treatment comprising administering to the subject the compound of Formula (I). The present invention is also directed to a novel method of preventing or treating infectious diseases and infectious disease-related complications in a subject that is in need of such prevention or treatment comprising administering to the subject the compound of Formula (I) and one or more antimicrobial agents.

In accordance with one embodiment of the present invention, there is provided a compound having the structure of Formula I:

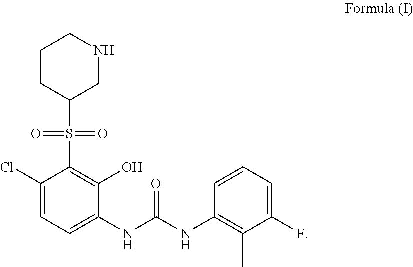 [SMILES: Cc1c(F)cccc1NC(=O)Nc1ccc(Cl)c(S(=O)(=O)C2CCCNC2)c1O]

In other embodiments, the compound of Formula (I) can also be depicted with its stereochemistry shown. Thus, the compound of Formula (I) is also a chiral compound having the structure:

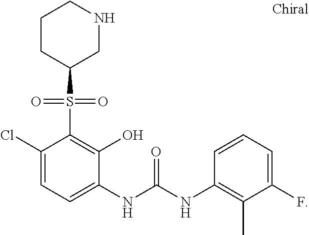 [SMILES: Cc1c(F)cccc1NC(=O)Nc1ccc(Cl)c(S(=O)(=O)[C@H]2CCCNC2)c1O]

The compound of Formula (I) is a CXCR2 inhibitor currently in Phase 2 clinical trials in the United States for Chronic Obstructive Pulmonary Disease (COPD) and referred to as “Danirixin” and by the chemical name: N-[4-chloro-2-hydroxy-3-(3-piperidinylsulfonyl)-pheny]-N′-(3-fluoro-2-methylphenyl)urea all of which can be referred to interchangeably herein. The compound of Formula (I) is described in U.S. Pat. No. 7,893,089, which patent is hereby incorporated by reference in its entirety.

In an alternate embodiment, there is also provided the compound of Formula I in the form of a hydrobromide salt as a standalone novel compound. In addition, such hydrobromide salt of the compound of Formula I may be used with the novel therapies and combinations of the present invention.

In another embodiment of the present invention, there is provided a combination treatment or preventative therapy comprising the compound of Formula (I) in combination with an antimicrobial agent. In one embodiment of the present invention, the antimicrobial agent is a neuraminidase inhibitor. In another embodiment of the present invention, the antimicrobial agent is selected from the group consisting of zanamivir, oseltamivir, laninamivir and peramivir. In yet another embodiment of the present invention, the antimicrobial agent is zanamivir. In a further embodiment of the present invention, the antimicrobial agent is oseltamivir. In one embodiment of the present invention, the antimicrobial agent is ribavirin.

Zanamivir is a marketed influenza virus neuraminidase inhibitor, known as Relenza®, and is approved by the United States FDA for the treatment and prophylaxis of influenza. See Ryan, D. M. et al., Antimicrob. Agents Chemother. 1994, 38, 2270. Zanamivir is dosed to a patient as a powder for inhalation at a 5 mg strength for use in a Diskhaler™ device. Zanamivir subsequently binds to the active site of the influenza neuraminidase enzyme, thus rendering the influenza virus unable to escape its host cell and infect others. Nevertheless, alternate modes of administration and alternate dosages of zanamivir are contemplated by the present invention, such as, for example, intravenous dosing.

Zanamivir has the following chemical structure:

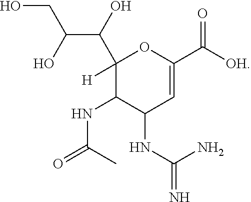 [SMILES: CC(=O)NC1C(NC(=N)N)C=C(C(=O)O)OC1C(O)C(O)CO]

In other embodiments, zanamivir can also be depicted with its actual stereochemistry shown. Such stereochemistry indicates that zanamivir is a chiral compound having the structure:

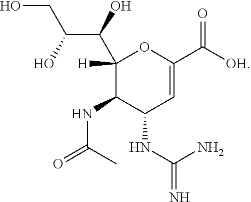 [SMILES: CC(=O)N[C@H]1[C@H]([C@H](O)[C@H](O)CO)OC(C(=O)O)=C[C@@H]1NC(=N)N]

Zanamivir is described in U.S. Pat. No. 5,360,817 to von Izstein, et al.; U.S. Pat. No. 5,597,933; U.S. Pat. No. 5,495,027; and U.S. Pat. No. 6,156,544, which patents are hereby incorporated by reference in their entirety. In addition to the disclosure in these patents, another synthesis route to make zanamivir has been reported. See Zhu, et al., Tetrahedron, 68(8), 2041-2044 (2012).

Oseltamivir is a marketed influenza virus neuraminidase inhibitor, known as Tamiflu®, and is approved by the United States FDA for the treatment and prophylaxis of influenza. See Lew, et al., Curr. Med. Chem., 7(6): 663-72 (2000).

Oseltamivir is dosed to a patient as capsules (containing oseltamivir phosphate 98.5 mg equivalent to oseltamivir 75 mg) and as a powder for oral suspension (oseltamivir phosphate equivalent to oseltamivir 6 mg/ml). Oseltamivir subsequently binds to the active site of the influenza neuraminidase enzyme, rendering the influenza virus unable to escape its host cell and infect others. Tamiflu® also is available in capsules containing 30 mg or 45 mg of Oseltamivir.

Oseltamivir has the following chemical structure:

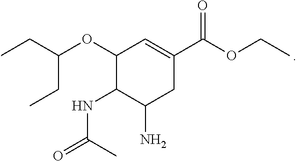 [SMILES: CCOC(=O)C1=CC(OC(CC)CC)C(NC(C)=O)C(N)C1]

In other embodiments, oseltamivir can also be depicted with its actual stereochemistry shown. Such stereochemistry indicates that oseltamivir is a chiral compound having the structure:

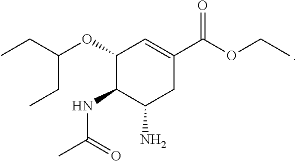 [SMILES: CCOC(=O)C1=C[C@@H](OC(CC)CC)[C@H](NC(C)=O)[C@@H](N)C1]

Oseltamivir is described in U.S. Pat. Nos. 5,763,483; 5,866,601; and 5,952,375; which patents are hereby incorporated by reference in their entirety. In addition to the disclosure in these patents, another synthesis route to make oseltamivir has been reported. See Ishikawa, et al., Angew. Chem. Int. Ed., 48: 1304-1307 (2009).

Therefore, in accordance with one embodiment of the present invention, there is provided a novel combination treatment therapy for a respiratory infection.

The present invention also provides a novel composition comprising the compound of Formula (I) in combination with zanamivir. In another embodiment, the present invention provides a novel composition comprising the compound of Formula (I) in combination with oseltamivir. In yet another embodiment, the present invention provides a novel composition comprising the compound of Formula (I) in combination with laninamivir. In yet another embodiment, the present invention provides a novel composition comprising the compound of Formula (I) in combination with peramivir. In yet another embodiment, the present invention provides a novel composition comprising the compound of Formula (I) in combination with favipiravir (T-705).

Further provided is a novel method of treating a respiratory infection in a subject suffering from the respiratory infection comprising administering to the subject the compound of Formula (I), or a pharmaceutically acceptable salt thereof, in combination with zanamivir, or a pharmaceutically acceptable salt thereof. Such “combinations” of the compound of Formula (I) and zanamivir can administered to a subject suffering from a respiratory infection as a fixed dose combination in the same dose, or such combinations can be administered in two separate doses.

Also provided are pharmaceutical compositions comprising a pharmaceutically acceptable carrier or excipient and the compound of Formula (I), or a pharmaceutically acceptable salt thereof, in combination with zanamivir, or a pharmaceutically acceptable salt thereof.

Also provided are methods of preventing a respiratory infection in a subject comprising administering to a subject at risk of, or predisposed to, acquiring a respiratory infection, the compound of Formula (I), or a pharmaceutically acceptable salt thereof, in combination with zanamivir, or a pharmaceutically acceptable salt thereof.

Further provided is a novel method of treating a viral respiratory infection in a subject suffering from the viral respiratory infection comprising administering to the subject the compound of Formula (I), or a pharmaceutically acceptable salt thereof, in combination with zanamivir, or a pharmaceutically acceptable salt thereof.

Further provided is a novel method of treating an influenza infection in a subject suffering from the influenza infection comprising administering to the subject the compound of Formula (I), or a pharmaceutically acceptable salt thereof, in combination with zanamivir, or a pharmaceutically acceptable salt thereof.

Further provided is a novel composition and/or method for treating an RSV infection in a subject suffering from the RSV infection comprising administering to the subject the compound of Formula (I), or a pharmaceutically acceptable salt thereof, in combination with Ribavirin, or a pharmaceutically acceptable salt thereof.

Such compounds of the present invention can exist in particular geometric or stereoisomeric forms. The invention contemplates all such compounds, including cis- and trans-isomers, (−)- and (+)-enantiomers, (R)- and (S)-enantiomers, diastereomers, (D)-isomers, (L)-isomers, the racemic mixtures thereof, and other mixtures thereof, such as enantiomerically or diastereomerically enriched mixtures, as falling within the scope of the invention. Additional asymmetric carbon atoms can be present in a substituent such as an alkyl group. All such isomers, as well as mixtures thereof, are intended to be included in this invention.

Optically active (R)- and (S)-isomers and d and I isomers can be prepared using chiral synthons or chiral reagents, or resolved using conventional techniques. If, for instance, a particular enantiomer of a compound of the present invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

In another embodiment of the invention, there is provided the compound of Formula (I) in combination with an antimicrobial agent, wherein the compound and antimicrobial agent is used in the manufacture of a medicament for use in the treatment of a viral infection in a human.

In another embodiment of the invention, there is provided a pharmaceutical composition comprising a pharmaceutically acceptable diluent and a therapeutically effective amount of a compound as defined in Formula (I) in combination with an antimicrobial agent.

In one embodiment, the present invention is directed to compounds, compositions and pharmaceutical compositions that have utility as novel treatments and/or preventative therapies for virus infections. In another embodiment, the present invention is directed to compounds, compositions and pharmaceutical compositions that have utility as novel treatments and/or preventative therapies for respiratory viral infections. In another embodiment, the present invention is directed to compounds, compositions and pharmaceutical compositions that have utility as novel treatments and/or preventative therapies for bacterial respiratory infections.

Viruses are classified by evaluating several characteristics, including the type of viral genome. Viral genomes can be comprised of DNA or RNA, can be double-stranded or single-stranded (which can further be positive-sense or negative-sense), and can vary greatly by size and genomic organization.

An RNA virus is a virus that has RNA (ribonucleic acid) as its genetic material. This nucleic acid is usually single-stranded RNA (ssRNA). RNA viruses can be further classified according to the sense or polarity of their RNA into negative-sense and positive-sense. Positive-sense viral RNA is similar to mRNA and thus can be immediately translated by the host cell. Negative-sense viral RNA is complementary to mRNA and thus must be converted to positive-sense RNA by an RNA polymerase before translation. As such, purified RNA of a positive-sense virus can directly cause infection though it may be less infectious than the whole virus particle. Purified RNA of a negative-sense virus is not infectious by itself as it needs to be transcribed into positive-sense RNA; each virion can be transcribed to several positive-sense RNAs.

Positive-sense, single-stranded RNA viruses (“positive-strand RNA viruses”) make up a large superfamily of viruses from many distinct subfamilies. These viruses span both the plant and animal kingdoms causing pathologies ranging from mild phenotypes to severe debilitating disease. The composition of the positive strand RNA virus polymerase supergroup includes, at least, the following families: levi-, narna-, picorna-, dicistro-, marna-, sequi-, como-, poty-, calici-, astro-, noda-, tetra-, luteo-, tombus-, corona-, arteri-, roni-, flavi-, toga-, bromo-, tymo-, clostero-, flexi-, seco-, barna, ifla-, sadwa-, chera-, hepe-, sobemo-, umbra-, tobamo-, tobra-, hordei-, furo-, pomo-, peclu-, beny-, ourmia-, and idaeovirus.

Negative-sense, single-stranded RNA viruses (“negative-strand RNA viruses”) must have their genome copied by an RNA-dependent RNA polymerase to form positive-sense RNA. This means that the virus must bring along with it the RNA replicase enzyme. The positive-sense RNA molecule then acts as viral mRNA, which is translated into proteins by the host ribosomes. The resultant protein goes on to direct the synthesis of new virions, such as capsid proteins and RNA replicase, which is used to produce new negative-sense RNA molecules.

There are eight families recognized in negative-sense single stranded RNA virus group and some unassigned to a particular family.
Order Mononegavirales
   Family Bornaviridae—Borna disease virus
   Family Filoviridae—includes Ebola virus, Marburg virus
   Family Paramyxoviridae—includes Measles virus, Mumps virus, Nipah virus, Hendra virus
   Family Rhabdoviridae—includes Rabies virus
Unassigned families:
   Family Arenaviridae—includes Lassa virus
   Family Bunyaviridae—includes Hantavirus, Crimean-Congo hemorrhagic fever
   Family Ophioviridae
   Family Orthomyxoviridae—includes Influenza viruses
Unassigned genera:
   Genus Deltavirus—includes Hepatitis D virus
   Genus Dichorhavirus
   Genus Emaravirus
   Genus Nyavirus—includes Nyamanini and Midway viruses
   Genus Tenuivirus
   Genus Varicosavirus
Unassigned species:
   Taastrup virus

Therefore, it is intended that the present invention can encompass the treatment or prevention of any of the viruses or families or genus of viruses recited herein and also additional viruses that are not recited herein, but yet would be known to one of skill in the art.

In one embodiment of the present invention, the compounds described herein are useful for preventing or treating viral infections in a subject caused by a single-stranded RNA virus.

In one embodiment of the present invention, the compounds described herein are useful for preventing or treating viral infections in a subject caused by a positive-sense, single-stranded RNA virus.

In one embodiment of the present invention, the compounds described herein are useful for preventing or treating viral infections in a subject caused by a negative-sense, single-stranded RNA virus.

In some embodiments, provided is a method for treating a viral infection in a subject mediated at least in part by a virus in the nidovirales, picornavirales, tymovirales, mononegavirales, reoviridae, pycobirnaviridae, parvoviridae, adenoviridae, poxviridae, polyomaviridae, herpesviridae, paramyxoviridae family of viruses, comprising administering to the subject a composition comprising a compound of any of Formula (I) in combination with an antimicrobial agent.

A method of treating a virus infection in a subject suffering from the virus infection comprising administering to the subject the compound of Formula (I) in combination with an antimicrobial agent.

A method of preventing a virus infection in a subject comprising administering to the subject a compound of any of Formula (I) in combination with an antimicrobial agent.

In other embodiments, the compounds described herein are useful for preventing or treating viral infections in a subject where the infection is caused by a virus belonging to the following families: levi-, narna-, picorna-, dicistro-, marna-, sequi-, como-, poty-, calici-, astro-, noda-, tetra-, luteo-, tombus-, corona-, arteri-, roni-, flavi-, toga-, bromo-, tymo-, clostero-, flexi-, seco-, barna, ifla-, sadwa-, chera-, hepe-, sobemo-, umbra-, tobamo-, tobra-, hordei-, furo-, pomo-, peclu-, beny-, ourmia-, and idaeovirus.

Compounds, methods and pharmaceutical compositions for treating respiratory viral infections, by administering to a subject having said viral infection the compound of Formula (I), alone or in combination with an antimicrobial agent, described herein, are disclosed. Methods for preparing such compounds and methods of using the compounds and pharmaceutical compositions thereof are also disclosed. In particular, the treatment and prophylaxis of viral infections such as those caused by RNA or DNA viruses are disclosed.

In other embodiments, the compounds described herein are useful for treating viral infections in a subject where the infection is caused by a virus belonging to the picornaviridae family, filoviridae family, paramyxoviridae family, or coronaviridae family. In other embodiments, the compounds described herein are useful for treating viral infections in a subject where the infection is caused by a virus belonging to the picornaviridae family. In other embodiments, the compounds described herein are useful for treating viral infections in a subject where the infection is caused by a virus belonging to the coronaviridae family.

In other embodiments, the compounds described herein are useful for preventing or treating viral infections in a subject where the infection is caused by any one or more viruses selected from the group consisting of poliovirus, rhinovirus, coxsackievirus, influenza A virus, influenza B virus, adenovirus, coronavirus, hepatitis A virus, hepatitis B virus, hepatitis C virus, hepatitis E virus, ebola virus, Marburg virus, Severe Acute Respiratory Syndrome (SARS) virus, arenavirus, Rift Valley Fever virus, yellow fever virus, respiratory syncytial virus (RSV), hepacivirus, west nile virus, Dengue fever virus, Aichi virus, enterovirus, rubella virus, murine encephalomyelitis virus, parainfluenza, metapneumovirus, foot-and-mouth virus, avian influenza virus and Middle East Respiratory Syndrome (MERS).

In yet other embodiments, the compounds described herein are useful for preventing or treating viral infections from any phylogenetic order, genus, family or particular species listed in Table 1 below.

TABLE 1
Positive-sense single stranded RNA viruses

Order Nidovirales

Family Arteriviridae
Family Coronaviridae - includes Coronavirus, SARS
Family Roniviridae

Order Picornavirales

Family Bacillariornaviridae
Family Caliciviridae - includes Norwalk virus
Family Dicistroviridae
Family flaviridae
Family Labyrnaviridae
Family Marnaviridae
Family Picornaviridae - includes Poliovirus, the “common cold” virus
(Rhinovirus), Hepatitis A virus, Coxsackievirus
Family Potyviridae
Family Secoviridae includes subfamily Comovirinae
Family Sequiviridae

Order Tymovirales

Family Alphaflexiviridae
Family Betaflexiviridae
Family Gammaflexiviridae
Family Tymoviridae

Unassigned

Family Alvernaviridae
Family Astroviridae
Family Barnaviridae
Family Bromoviridae
Family Closteroviridae
Family Flaviviridae - includes Yellow fever virus, West Nile virus, Hepatitis
C virus, Dengue fever virus
Family Leviviridae
Family Luteoviridae
Family Narnaviridae
Family Nodaviridae
Family Retroviridae - includes human immunodeficiency virus 1 and 2
Family Tetraviridae
Family Togaviridae - includes Rubella virus, Ross River virus, Sindbis
virus, Chikungunya virus
Family Tombusviridae
Family Virgaviridae

Negative-sense single stranded RNA viruses

Order Mononegavirales

Family Bornaviridae - Borna disease virus
Family Filoviridae - includes Ebola virus, Marburg virus
Family Paramyxoviridae - includes Measles virus, Mumps virus, Nipah
virus, Hendra virus, respiratory syncytial virus (RSV), human
parainfluenza viruses (PIVs), human metapneumovirus (hMPV)
Family Rhabdoviridae - includes Rabies virus

Unassigned families:

Family Arenaviridae - includes Lassa virus, Junin virus
Family Bunyaviridae - includes Hantavirus, Crimean-Congo hemorrhagic
fever
Family Ophioviridae
Family Orthomyxoviridae - includes Influenza viruses

Unassigned genera:

Genus Deltavirus
Genus Emaravirus
Genus Nyavirus - includes Nyamanini and Midway viruses

Double stranded RNA viruses

Family Reoviridae - includes Rotavirus
Family Pycobirnaviridae - includes human pycobirnavirus

DNA viruses

Family Parvoviridae - includes Parvovirus B19
Family Adenoviridae- includes adenovirus
Family Poxviridae - includes monkey pox
Family Polyomaviridae - includes BK virus
Family Herpesviridae - includes herpes simplex virus

In other embodiments, the compounds described herein are useful for treating viral infections in a subject where the infection is caused by a virus belonging to the paramyxoviridae family, picornaviridae family, or flaviviridae family. In other embodiments, the compounds described herein are useful for treating viral infections in a subject where the infection is caused by a virus belonging to the paramyxoviridae family. In other embodiments, the compounds described herein are useful for treating viral infections in a subject where the infection is caused by a virus belonging to the flaviviridae family.

In other embodiments, the compounds described herein are useful for treating viral infections in a subject where the infection is caused by a virus belonging to the picornaviridae family.

In other embodiments, the compounds described herein are useful for treating viral infections in a subject where the infection is caused by any one or more viruses selected from the group consisting of poliovirus, rhinovirus, coxsackievirus, influenza A virus, influenza B virus, influenza C virus, adenovirus, coronavirus, hepatitis A virus, hepatitis B virus, hepatitis C virus, hepatitis E virus, ebola virus, Marburg virus, Severe Acute Respiratory Syndrome (SARS) virus, arenavirus, Rift Valley Fever virus, yellow fever virus, respiratory syncytial virus (RSV), west nile virus, Dengue fever virus, Aichi virus, enterovirus, rubella virus, Theiler's murine encephalomyelitis virus (TMEV), foot-and-mouth virus (FMDV), human immunodeficiency virus (HIV), respiratory syncytial virus (RSV), parainfluenza virus (PIV), human PIVs, human metapneumovirus (hMPV), avian influenza virus, and Middle East Respiratory Syndrome (MERS).

In other embodiments, the compounds described herein are useful for treating viral infections in a subject where the infection is caused by any of the human enteroviruses A-D.

In other embodiments, the compounds described herein are useful for treating viral infections in a subject where the infection is caused by enterovirus A71.

In other embodiments, the compounds described herein are useful for treating viral infections in a subject where the infection is caused by any of the human rhinoviruses A-C.

In other embodiments, the compounds described herein are useful for treating viral infections in a subject where the infection is caused by human rhinovirus A.

In other embodiments, the compounds described herein are useful for treating viral infections in a subject where the infection is caused by human rhinovirus B.

In other embodiments, the compounds described herein are useful for treating viral infections in a subject where the infection is caused by human rhinovirus C.

In other embodiments, the compounds described herein are useful for treating viral infections in a subject where the infection is caused by human respiratory syncytial virus.

In other embodiments, the compounds described herein are useful for treating viral infections in a subject where the infection is caused by human respiratory syncytial virus A.

In other embodiments, the compounds described herein are useful for treating viral infections in a subject where the infection is caused by human respiratory syncytial virus B.

In other embodiments, the compounds described herein are useful for treating viral infections in a subject where the infection is caused by the Aichi virus.

In other embodiments, the compounds described herein are useful for treating viral infections in a subject where the infection is caused by the poliovirus.

In other embodiments, the compounds described herein are useful for treating viral infections in a subject where the infection is caused by the coxackievirus.

In other embodiments, the compounds described herein are useful for treating viral infections in a subject where the infection is caused by the echovirus.

In other embodiments, the compounds described herein are useful for treating viral infections in a subject where the infection is caused by the hepatitis A virus.

In other embodiments, the compounds described herein are useful for treating viral infections in a subject where the infection is caused by the severe acute respiratory syndrome virus.

In other embodiments, the compounds described herein are useful for treating viral infections in a subject where the infection is caused by the Juninvirus.

In other embodiments, the compounds described herein are useful for treating viral infections in a subject where the infection is caused by the monkey pox virus.

In other embodiments, the compounds described herein are useful for treating viral infections in a subject where the infection is caused by the rift valley fever virus.

In other embodiments, the compounds described herein are useful for treating viral infections in a subject where the infection is caused by the hepatitis B virus.

In other embodiments, the compounds described herein are useful for treating viral infections in a subject where the infection is caused by the hepatitis C virus.

In other embodiments, the compounds described herein are useful for treating viral infections in a subject where the infection is caused by the human immunodeficiency virus (HIV)

In other embodiments, the compounds described herein are useful for treating viral infections in a subject where the infection is caused by the influenza virus.

In other embodiments, the compounds described herein are useful for treating viral infections in a subject where the infection is caused by the influenza A virus.

In other embodiments, the compounds described herein are useful for treating viral infections in a subject where the infection is caused by the influenza B virus.

In other embodiments, the compounds described herein are useful for treating viral infections in a subject where the infection is caused by the influenza C virus.

In other embodiments, the compounds described herein are useful for treating viral infections in a subject where the infection is caused by a coronavirus.

In other embodiments, the compounds described herein are useful for treating viral infections in a subject where the infection is caused by a virus belonging to the filoviridae family.

In other embodiments, the compounds described herein are useful for treating viral infections in a subject where the infection is caused by a virus belonging to the arenaviriade family.

In other embodiments, the compounds described herein are useful for treating viral infections in a subject where the infection is caused by a virus belonging to the bunyaviridae family.

In other embodiments, the compounds described herein are useful for treating viral infections in a subject where the infection is caused by human immunodeficiency virus 1 and/or human immunodeficiency virus 2.

### Synthesis of the Compound of Formula (I)

The following example serves to more fully describe the manner of making the compound of Formula (I). One of skill in the art will appreciate how to synthesize the compound of Formula (I) after reading United States Patent No. 7,893,089, which is fully incorporated herein by reference.

It is understood that this example in no way serves to limit the true scope of the invention, but rather are presented for illustrative purposes.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When desired, the (R)- and (S)-isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation.

These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

All references to ether are to diethyl ether; brine refers to a saturated aqueous solution of NaCl, DCM refers to dichloromethane, THF refers to tetrahydrofuran, EtOAc refers to ethyl acetate, Hex and Hx refers to hexane, IMS refers to industrial methylated spirit, TBME refers to tert-butylmethyl ether, DMF refers to dimethylformamide, BOC and Boc refers to tert-butyloxycarbonyl. Unless otherwise indicated, all temperatures are expressed in ° C. (degrees Centigrade). All reactions are conducted under an inert atmosphere at room temperature unless otherwise noted.

¹H NMR spectra were recorded on a Jeol Delta2 (300 MHz) spectrometer. Chemical shifts are expressed in parts per million (ppm, δ units). Splitting patterns describe apparent multiplicities and are designated as s (singlet), d (doublet), t (triplet), q (quartet), quint (quintet), m (multiplet), br (broad).

Unless otherwise stated, “flash” and “column chromatography” refers to flash column chromatography on silica using the stated solvent systems. LC-MS data were obtained on either a PE Sciex Single Quadrupole LC/MS API-150 combined with a Shimadzu LC system (SCL-10A Controller and dual UV detector) or on a Waters micromass ZQ combined with a Waters 2695 separation module.

### Starting Material 1

### N-(3,4-dichlorophenyl)-2,2-dimethylpropanamide

3,4-dichloroaniline (150 g) was dissolved in 1.0 L TBME and the solution was cooled to 10° C. Sodium hydroxide (140.7 g of a 30% aqueous solution) was added under mechanical stirring. Pivaloyl chloride (125.9 mL) was added dropwise while keeping the internal temperature under 35° C. After the addition, the temperature of the reaction was maintained at 30-35° C. for a further 30 min. The reaction mixture was then allowed to cool to room temperature and subsequently kept at 0-5° C. for 1 h. The resulting precipitate was filtered of and washed with 600 mL water/MeOH (90/10) and then with 900 mL water. The resulting solid was the dried in a vacuum oven at 55° C. for 4 days. Yield: 162 g. ¹H-NMR (DMSO-d₆) δ9.46(s, 1H), 8.04 (d, J=2.4 Hz, 1H), 7.65 (dd, J=9.0. 2.4 Hz, 1H), 7.54 (d, J=9.0 Hz, 1H), 1.22 (9H, s).

### Starting Material 2

### 6-chloro-2-(1,1-dimethylethyl)-1,3-benzoxazole-7-sulfonyl chloride

N-(3,4-dichlorophenyl)-2,2-dimethylpropanamide (121 g) was dissolved in 720 mL THF and the solution was cooled to −50° C. Butyllithium (433 mL, 2.5N in hex) was added while keeping the internal temperature between −45° C. and −35° C. (final temp.: −35° C.). Held at −25° C. for 40 min. An HPLC check of the reaction mixture revealed that 5-10% of the starting material remained. An additional 35 mL of butyllithium was added at −30° C. and the reaction was at −30 to -25° C. for a further 30 min (HPLC: no significant change). The reaction mixture was cooled to −45° C. and SO₂ was bubbled though the solution until saturation appeared to have been reached. Subsequently, the reaction mixture was stirred at −10 to 0° C. for 45 min. Argon (2 double-balloon volumes) was bubbled through the solution following which the reaction mixture was cooled to −5° C. Sulfuryl chloride (58.8 mL) was added while keeping the temperature below 22° C. Subsequently, the reaction mixture was kept at 10-15° C. for 1 h (HPLC: complete). EtOAc was added and the mixture was concentrated, washed with water, saturated aqueous sodium bicarbonate and brine, dried over MgSO₄ and the solvent was evaporated in vacuo. The crude material crystallized and was triturated with hot hexane. Yield: 87.2 g ¹H-NMR (DMSO-d₆) δ7.60(d, J=8.4 Hz, 1H), 7.34(d, J=8.4 Hz, 1H), 1.43(9H, s).

### Intermediate 1

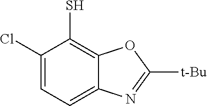 [SMILES: CC(C)(C)c1nc2ccc(Cl)c(S)c2o1]

Starting Material 1, N-(3,4-dichlorophenyl)-2,2-dimethylpropanamide (prepared according to WO01/68033A2, incorporated herein by reference, to the extent that it teaches the synthesis of Starting Material 1, also described above) was dissolved in dry THF (400 mL), then cooled to −75° C.) under an argon atmosphere. n-BuLi (160 mL, 2.5M in hexane, 5 eq.) was added dropwise while keeping the temperature below −60° C. Once all the n-BuLi was added, the reaction was stirred at −5° C. for 1.5 h, then cooled to −70° C. and sulfur (“sulfur flowers”) (13 g) was added followed by stirring at −70° C. to room temperature overnight. After stirring the reaction mixture at −10° C., the solution changed color from yellow to brown/orange. The reaction mixture was cooled to 0° C., then quenched with 2N HCl solution (200 mL) and stirred for 10 min. The organic layer was separated and basified with 2N NaOH solution to pH 12-13, then washed with EtOAc. The aqueous layer was reacidified with 2M HCl solution to about pH 1 and extracted with dichloromethane (2X) which was washed with water, dried over Na₂SO₄ and concentrated. The crude product was purified by column chromatography using 1:5 (EtOAc/Hex). Yield: 6 g (30%, orange oil). ¹H-NMR (CDCl₃) δ7.39-7.30 (m, 2H), 4.08 (s, 1H), 1.50 (9H, s).

Alternatively, Intermediate 1 is prepared in the following way: Triphenylphosphine (89 g) was dissolved in DCM (200 ml) and DMF (2.2 ml). The solution was cooled in an ice/methanol bath to −1° C. To this was added a solution of the 6-chloro-2-(1,1-dimethylethyl)-1,3-benzoxazole-7-sulfonyl chloride, Starting Material 2, (prepared according to WO01/68033A2, incorporated herein by reference, to the extent that it teaches the synthesis of Starting Material 2, also described above) (35 g) in DCM (100 ml) over 30 minutes maintaining the temperature below 15° C. The reaction mixture was stirred at room temperature under nitrogen for 18 hours. The reaction mixture was quenched using 2N hydrochloric acid (200 ml). The phases were separated and the organic phase was evaporated in vacuo. The residue was suspended in 2N sodium hydroxide (400 ml) and stirred rapidly for 3 hours. The solid was removed by filtration and washed with water. The combined filtrate and washings were cooled in an ice/water bath and acidified using 5N hydrochloric acid to ˜pH 1. This was extracted using TBME (400 ml). The organic phase was dried over magnesium sulfate and evaporated in vacuo to give Intermediate 1 (22.85 g) as a brown solid.

### Intermediate 2: (General procedure A)

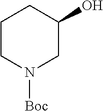 [SMILES: CC(C)(C)OC(=O)N1CCC[C@@H](O)C1]

To a suspension of (R)-(+)-3-hydroyxpiperidine hydrochloride (1 g) in DCM (20 mL) was added Et₃N (3.04 mL) followed by BOC₂O (1.75 g) at 0° C. which was left over the weekend. Water (50 mL) was added and extracted with DCM (100 mL). Combined organics were washed with water (2×50 mL) then brine (50 mL), dried (Na₂SO₄) and concentrated. The residue was columned (flash, eluted with a gradient of 0-10% MeOH/DCM). Yield: 1.55 g. ¹H-NMR (CDCl₃) δ3.74-3.69 (2H, m), 3.56-3.48 (1H, m), 3.18-3.03 (2H, m), 1.92-1.83 (1H, m), 1.79-1.71(2H, m), 1.55-1.45 (1H, m), 1.43 (9H, s).

### Intermediate 3: (General procedure B)

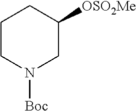

To a solution of Intermediate 2 (1 g) in DCM (10 mL) was added Et₃N (1.38 mL) followed by MsCl (0.46 mL) dropwise at 0° C. After stirring at 0° C. for 1 hour the reaction was warmed to room temperature, quenched with water (10 mL) and separated. The aqueous layer was extracted with DCM (2×20 mL). Combined organics were washed with water (40 mL), a spatula of silica added, dried (NaSO₄) and concentrated. Yield: 1.4148 g. ¹H-NMR (CDCl₃) δ4.71 (1 H, br s), 3.62 (2H, br d), 3.49-3.27 (2H, m), 3.04 (3H, s), 2.01-1.76 (3H, m), 1.79-1.71(2H, m), 1.55-1.45 (1H, m), 1.45 (9H, s).

### Intermediate 4: (General procedure C)

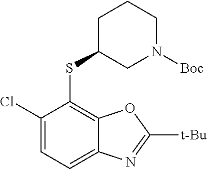 [SMILES: CC(C)(C)OC(=O)N1CCC[C@H](Sc2c(Cl)ccc3nc(C(C)(C)C)oc23)C1]

To a suspension of NaH (0.30 g) in THF (20 mL) was added Intermediate 1 (using Starting Material 1) (1.22 g) dropwise. After stirring for 1 hour, Intermediate 3 (1.41 g) in THF was added and the reaction heated to 80° C. and left overnight. The reaction mixture was cooled to room temperature then quenched with aqueous saturated NaHCO₃ (50 mL). Reaction mixture was extracted with DCM (2×50 mL). Combined organics were washed with water (100 mL), dried (NaSO₄) and concentrated. Residue columned (flash, 20% EtOAC/Hx, silica). Yield: 946.9 mg. ¹H-NMR (CDCl₃) δ7.50 (d, J=7.9 Hz, 1H), 7.38 (d, J=7.9 Hz, 1H), 3.82 (d, J=13.4 Hz, 1H), 3.55-3.45 (m, 1H), 3.00-2.80 (m, 2H).

### Intermediate 5: (General procedure D)

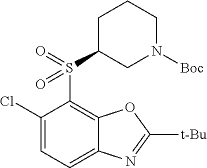 [SMILES: CC(C)(C)OC(=O)N1CCC[C@H](S(=O)(=O)c2c(Cl)ccc3nc(C(C)(C)C)oc23)C1]

To a solution of intermediate 4 (946.9 mg) in DCM (10 mL) was added mCPBA (2.31 g) in DCM (10 mL) at −10° C. The reaction was stirred at −10° C. for 1 h, then warmed to room temperature. The reaction mixture was quenched with aqueous saturated NaHCO₃ (50 mL) then extracted with DCM (2×70 mL). Combined organics were washed with water (50 mL), dried (Na₂SO₄) and concentrated. Residue columned (flash, 30% EtOAc/Hx, silica). Yield 353.6 mg (35%, yellow oil). MS (m/z, ES⁺, M+H): 457.08.

### Intermediate 6: (General procedure E)

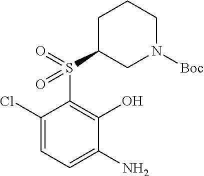 [SMILES: CC(C)(C)OC(=O)N1CCC[C@H](S(=O)(=O)c2c(Cl)ccc(N)c2O)C1]

To a solution of Intermediate 5 (353 mg) in IMS (5 mL) was added aqueous concentrated HCl(5 mL). The reaction was then heated to 80° C. and left overnight. Reaction mixture was cooled to room temperature and was concentrated to remove IMS. Residue was basified to pH 12 with aqueous saturated NaOH, EtOAc (30 mL), BOC₂O (1 eq., 0.17 g) added at 0° C. and left overnight. Reaction mixture was separated, and aqueous layer extracted with EtOAc (2×30 mL). Combined organics were dried (with Na₂SO₄) and concentrated. Residue was columned (flash, eluted with a gradient of 10% -30% EA/Hx). Yield: Two product containing fractions were isolated: 58.0 mg and 180.9 mg. MS (m/z, ES⁺, M+H): 291.01.

### Intermediate 7: (General procedure F)

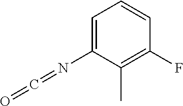

3-fluoro-2-methylaniline (7.4 g) was dissolved in DCM (220 mL) at room temperature under an argon atmosphere. After cooling to 0° C., aqueous saturated NaHCO₃ (220 mL) was added followed by triphosgene (5.85 g). The reaction was left to stir at 0° C. for 1 h. After this time, the product was extracted with DCM (2×50 mL). The organic fractions were combined, dried over MgSO₄ and the solvent removed in vacuo to yield a yellow oil. Addition of hexane allowed precipitation of a white salt which was filtered off. Removal of the hexane in vacuo yielded a yellow oil (7.69 g, 86%). ¹H-NMR (CDCl₃) δ7.09 (dd, 1H), 6.92-6.85 (m, 2H), 2.24 (s, 3H).

### Intermediate 8: (General procedure G)

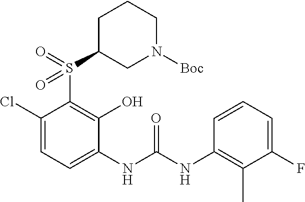 [SMILES: Cc1c(F)cccc1NC(=O)Nc1ccc(Cl)c(S(=O)(=O)[C@H]2CCCN(C(=O)OC(C)(C)C)C2)c1O]

To a solution of Intermediate 6 (60 mg) in DCM (3 mL) was added Intermediate 7 (70 mg) and the reaction was left over the weekend. Reaction mixture was concentrated and the residue columned (flash, eluted with a gradient of 20% -30% EtOAc/Hx). Yield: 56.2 mg. MS (m/z, ES⁺, M+H): 542.01.

### EXAMPLE 1

### N-{4-chloro-2-hydroxy-3-[(3S)-3-piperidi nylsulfonyl]phenyl}-N′-(3-fluoro-2-methylphenyl)urea. (General procedure H)

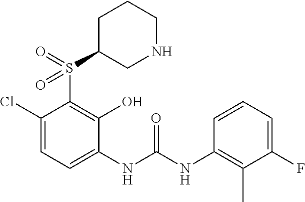 [SMILES: Cc1c(F)cccc1NC(=O)Nc1ccc(Cl)c(S(=O)(=O)[C@H]2CCCNC2)c1O]

Intermediate 8 (56.2 mg) and 4N HCl/dioxane (3 mL) were stirred together at room temperature and left overnight. Intermediates 6, 5, 4, 3 and 2 were made as described above. Intermediate 1 was made using Starting Material 1 for synthesizing Example 1. The reaction mixture was concentrated and residue dissolved in minimum amount of MeOH and Et₂O was added. Solid crashed out which was filtered and dried. Crude yield: 28.4 mg. The crude product was dissolved in a minimum amount of MeOH and Et₂O added. Solid crashed out, the solvent was decanted and solid dried. Yield: 18.8 mg. MS (m/z, ES⁺, M+H): 441.98. NMR (MeOD) δ8.40 (1H, d, ArH), 7.46 (1H, d, ArH), 7.19-7.15 (2H, m, ArH), 6.85 (1H, t, ArH), 4.14 (1H, dt, CH), 3.66 (1H, dd, CH), 3.37 (2H, d, CH₂), 3.04 (1H, dt, CH), 2.19 (3H, S, ArCH₃), 2.14-1.69 (4H, m, 2xCH₂).

One embodiment of the invention encompasses combinations comprising the compound of Formula (I) alone and/or in combination with one or more additional therapeutic agents. For example, in one embodiment, the invention encompasses a combination comprising the compound of Formula (I) in combination with one or more antimicrobial agents selected from those agents in Table 2, Table 3, and/or Table 4. In one embodiment, the antimicrobial agent is chosen from those antiviral agents found in Table 2.

TABLE 2

| Compound | | Chemical |
|---|---|---|
| Name | Structure | Name |
| Relenza ® (zanamivir) | 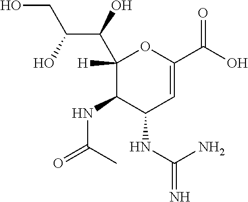 [SMILES: CC(=O)N[C@H]1[C@H]([C@H](O)[C@H](O)CO)OC(C(=O)O)=C[C@@H]1NC(=N)N] | (2R,3R,4S)-4-guanidino-3-(prop-1- en-2-ylamino)-2-((1R,2R)-1,2,3- trihydroxypropyl)-3,4-dihydro-2H- pyran-6-carboxylic acid |
| Tamiflu ® (oseltamivir) | 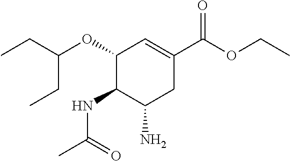 [SMILES: CCOC(=O)C1=C[C@@H](OC(CC)CC)[C@H](NC(C)=O)[C@@H](N)C1] | ethyl (3R,4R,5S)-5-amino-4- acetamido-3-(pentan-3-yloxy)- cyclohex-1-ene-1-carboxylate |
| Symmetrel ® (amantadine) | 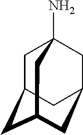 | adamantan-1-amine |
| Flumadine ® (rimantadine) | 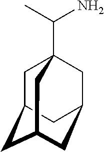 | (RS)-1-(1-adamantyl)ethanamine |
| Peramivir | 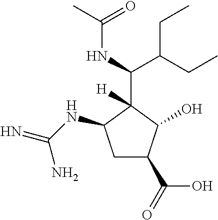 [SMILES: CCC(CC)[C@H](NC(C)=O)[C@@H]1[C@H](O)[C@@H](C(=O)O)C[C@H]1NC(=N)N] | (1S,2S,3S,4R)-3-[(1S)-1- acetamido-2-ethyl-butyl]-4- (diaminomethylideneamino)-2- hydroxy-cyclopentane-1- carboxylic acid |
| Laninamivir | 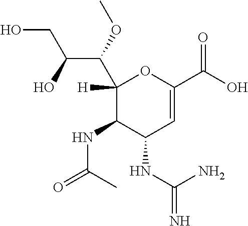 [SMILES: CO[C@H]([C@@H]1OC(C(=O)O)=C[C@H](NC(=N)N)[C@H]1NC(C)=O)[C@@H](O)CO] | (4S,5R,6R)-5-acetamido-4- carbamimidamido-6-[(1R,2R)-3- hydroxy-2-methoxypropyl]-5,6- dihydro-4H-pyran-2-carboxylic acid |
| Arbidol ™ (umifenovir) | 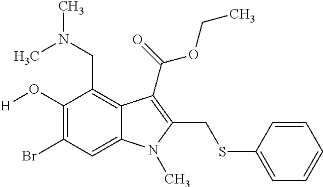 [SMILES: CCOC(=O)c1c(CSc2ccccc2)n(C)c2cc(Br)c(O)c(CN(C)C)c12] | 1-methyl-2-((phenylthio)methyl)-3- carbethoxy-4- ((dimethylamino)methyl)-5- hydroxy-6-bromindole |
| Favipiravir | 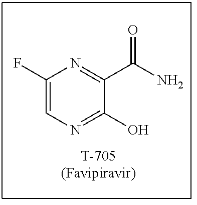 [SMILES: NC(=O)c1nc(F)cnc1O] | 6-fluoro-3-hydroxy-2- pyrazinecarboxamide |
| Ribavirin | 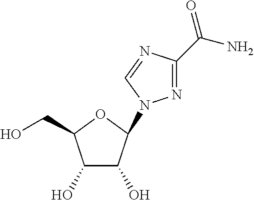 [SMILES: NC(=O)c1ncn([C@@H]2O[C@H](CO)[C@@H](O)[C@H]2O)n1] | 1-[(2R,3R,4S,5R)-3,4-dihydroxy-5- (hydroxymethyl)oxolan-2-yl]-1H- 1,2,4-triazole-3-carboxamide |
| Taribavirin Viramidine | 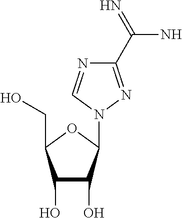 | 1-[(2R,3R,4S,5S)-3,4-dihydroxy- 5-(hydroxymethyl)oxolan-2-yl]- 1,2,4-triazole-3-carboximidamide |

In other embodiments of the present invention, the antiviral agent is chosen from acyclovir, gancyclovir, interferons, thimerasol, idoxuridine, vidarabine, trifluridine, famciclovir, valacyclovir, penciclovir, ganciclovir, dipyridamole, impulsin, pleconaril, foscarnet, cidofovir, ICI 130,685, valganciclovir, acyclovir, idoxuridine, vidarabine, or valacyclovir.

In one embodiment of the present invention, the antimicrobial agent is zanamivir.

Zanamivir is a marketed potent influenza virus neuraminidase inhibitor, known as Relenza®, and approved by the United States FDA for the treatment and prophylaxis of influenza.

The synthesis of zanamivir is described in Example 3 of U.S. Pat. No. 5,360,817 to von Izstein, et al., which patent is herein incorporated by reference in its entirety.

For instance, the process for preparation of zanamivir was described therein as a selective deacetylation of 5-acetamido-4-acetoxy-6-(I,2,3-triacetoxypropyl)-5,6-dihydro-4H-pyran-2-carboxylate of Formula (II) with boron trifluoride ethearate which gave 5-acetamido-4-hydroxy-6-(I,2,3-triacetoxypropyI)-5,6-dihydro-4H-pyran-2-carboxylate of Formula (III), which on further treatment with trifluoromethanesulfonic anhydride and sodium azide gave 5-acetamido-4-azido-6-(I,2,3-triacetoxypropyl)-5,6-dihydro-4H-pyran-2-carboxylate of Formula (IV). The reduction of intermediate compound of Formula (IV) with hydrogen sulphide in pyridine afforded the corresponding 5-acetamido-4-amino-6-(I,2,3-triacetoxypropyl)-5,6-dihydro-4H-pyran-2-carboxylate intermediate of Formula (V), which was finally condensed with S— methylisothiourea in water and saponified through Dowex 50W in aqueous ammonium hydroxide to yield zanamivir.

In still further embodiments of the present invention, the antimicrobial agent is an antibiotic. For purposes of the present invention, combinations of the compound of Formula (I) and an antimicrobial agent, such as an antibiotic, provides an effective treatment therapy subjects suffering from a respiratory bacterial infectious disease. The term “antibacterial” or “antibiotic” used interchangeably herein, means any chemical of natural or synthetic origin which has the effect to kill or inhibit or suppress the growth of biological cells. Examples of antibacterial agents encompassed by the combination methods and compositions of the present invention include those antibiotics and antibiotic classes set forth in table 3 below. See, Todar, K., Todar's Textbook of Bacteriology, University of Wisconsin-Madison, Department of Bacteriology (2002) and The Merck Manual, Sec. 13. Chap. 153., “Antibacterial Drugs,” 17^{th} Edition (1999).

TABLE 3
Table 3
Classes and Examples of Antibiotic Antimicrobial agents

| Antibiotic Class | Examples |
|---|---|
| Beta-lactams-penicillins | Penicillin G, Penicillin V, Procaine, Benzathine, Cloxacillin, |
| | Dicloxacillin, Methicillin, Nafcillin, Oxacillin, Azlocillin, Carbenicillin, |
| | Piperacillin, Piperacillin plus Tazobactam, Ticarcillin and Mezlocillin |
| Beta-lactams - | First-generation |
| Cephalosporins | Cefadroxil, Cefazolin, Cephalexin, Cephalothin, Cephapirin and |
| | Cephradine |
| | Second-generation |
| | Cefaclor, Cefamandole, Cefmetazole Cefonicid, Cefotetan, |
| | Cefoxitin, Cefprozil, Cefuroxime and Loracarbef |
| | Third-generation |
| | Cefepime, Cefixime, Cefoperazone, Cefotaxime, Cefpodoxime, |
| | Ceftazidime, Cefibuten, Ceftizoxime and Ceftriaxone |
| Other Beta-lactams | Meropenem, Sulbactam, Tazobactam |
| Semisynthetic penicillin | Ampicillin, Ampicillin plus Sulbactam, Amoxycillin, Amoxicillin, |
| | Amoxicillin plus clavulanate and Bacampicillin |
| Clavulanic acid | Clavamox (clavulanic acid plus amoxicillin) |
| Monobactams | Aztreonam |
| Aminoglycosides | Streptomycin, Kanamycin, Neomycin, Gentamycin, Tobramycin, |
| | Amikacin and Netilmicin |
| | Gentamicin |
| Glycopeptides | Vancomycin |
| Lincomycins | Clindamycin |
| Macrolides and Azalides | Azithromycin, Clarithromycin, Clindamycin, Erythromycin, |
| | Lincomycin, Roxithromycin, Dirithromycin, Spiramycin and |
| | Josamycin |
| Polypeptides | Bacitracin, Colistin, Polymyxin B |
| | Bacitracin |
| Rifamycins | Rifampicin |
| Tetracyclines | Tetracycline, Chlortetracycline, Oxytetracycline, Demeclocycline |
| | and Minocycline |
| Semisynthetic | Doxycycline |
| Tetracyclines | |
| Chloramphenicol | Chloramphenicol |
| Fluoroquinolones and | Ciprofloxacin (Cipro ®), Enoxacin, Grepafloxacin, Levofloxacin, |
| Quinolones | Lomefloxacin, Norfloxacin, Ofloxacin, Sparfloxacin, Trovafloxacin, |
| | Cinoxacin and Nalidixic acid |
| Lincosamides | Clindamycin (Cleocin ®) |
| Antibiotic Class | Examples |
| Oxazolidinones | Linezolid (Zybox ®) |
| Aminocyclitols | Spectinomycin (Trobicin ®) |
| Cycloserines | |
| Mupirocin | |
| Streptogramins | Quinupristin and dalfopristin (Synercid ®) |
| Urea hydroxamates | |
| Heteroaromatic polycycles | |
| Folic Acid Analogs | Trimethoprim and Trimethoprim-sulfamethoxazole (TMP-SMX) |
| Sulfa Drugs | Sulfanilamide, Sulfadiazine, sulfamethoxazole, Sulfisoxazole, |
| (sulfonamides) | Sulfamethizole, Silver sulfadiazine and Mafenide |

TABLE 4

| Name | Structure/Description | Company/Univ. | Dosage | Citation |
|---|---|---|---|---|
| Virazole (Ribavirin) 1-beta-D- ribofuranosyl- 1H-1,2,4- triazole-3- cart)oxamide | 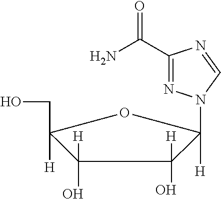 | U.S. Pat. No. 4,211,771 assigned 1980 to ICN Pharma; 1983 to Viratek, Inc. Currently sold by Valeant Pharma. | 20 mg/mL in small particle aersol generator (SPAG- 2) for 12-18 hrs/day for 3-7 days, delivering 190 ug/L air in 12 hrs. | U.S. Pat No. 4,211,771 - (process of treating w/drug). |
| ALN-RSV01 | siRNA comprised of 2 unpaired thymidine | Alnylam | 0.6 mg/kg | PNAS. 2010 |
| | overhangs + 19 nucleotides | Pharma | once/day for 3 days | May; 107(19): 8800-8805. |
| | complimentary to nucleotides 3-21 of the | Completed | in Phase IIb study. | U.S. Pat. No. 8,410,073, |
| | mRNA encoding RSV nucleocapsid N | phase IIb, | | US20090238772 |
| | protein. | missed primary | | |
| | | endpoint. | | |
| ALN-RSV02 | siRNA | Alnylam | | No references identified. |
| Benzimidazoles | 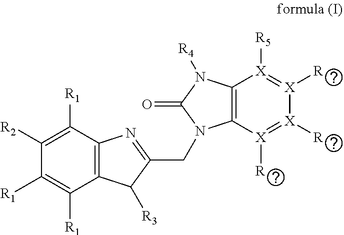 | U.S. Pat. No. 8,865,705 assigned to Janssen R&D Ireland | | U.S. Pat. No. 8,865,705 |
| BTA-9881 | Structure not reported | Biota Holdings; | | See Emerging |
| | | Medlmmune/ | | Drugs Reviews - |
| | | Astra Zeneca | | 2010, 2012. |
| BTA-C286 | | Biota | | |
| TMC353121 (derivative of JNJ-2408068) | 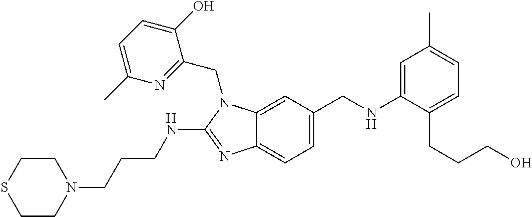 [SMILES: Cc1ccc(CCCO)c(NCc2ccc3nc(NCCCN4CCSCC4)n(Cc4nc(C)ccc4O)c3c2)c1] | Tibotec | | J. Med. Chem, 2008, 51, 875-896. Several apps list TMC353121 as a combination or as a substitute, such as US20110293521, US20110295365, US20110293686, US20110290821. \ |
| JNJ-2408068 (formerly R- 170591) | 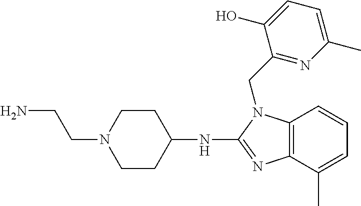 [SMILES: Cc1ccc(O)c(Cn2c(NC3CCN(CCN)CC3)nc3c(C)cccc32)n1] | Johnson & Johnson | | J. Med. Chem, 2007, 50, 4572-4584. |
| YM-53403 | 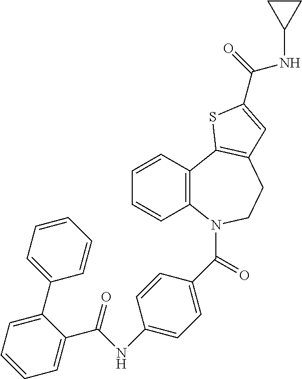 [SMILES: O=C(NC1CC1)c1cc2c(s1)-c1ccccc1N(C(=O)c1ccc(NC(=O)c3ccccc3-c3ccccc3)cc1)CC2] | Yamanouchi Pharma | | Antiviral Research 65 (2005) 125-131. |
| BMS-433771 (benzimidazole) | 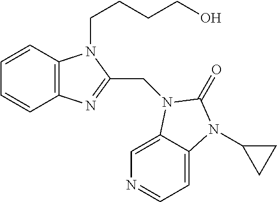 [SMILES: O=c1n(Cc2nc3ccccc3n2CCCCO)c2cnccc2n1C1CC1] | Bristol-Myers Squibb | | Combrink et al. Bioorg. Med. Chem. Lett. 17 (2007) 4784-90. |
| VX-497 | 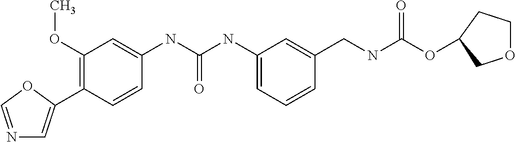 [SMILES: COc1cc(NC(=O)Nc2cccc(CNC(=O)O[C@H]3CCOC3)c2)ccc1-c1cnco1] | Vertex | | Antimicrob Agents Chemother., Apr. 2000, p. 859-866. WO 97/41211 and WO 01/00622 (assigned to Vertex). See US20050187170 |
| VP-14637; aka MDT-637 | 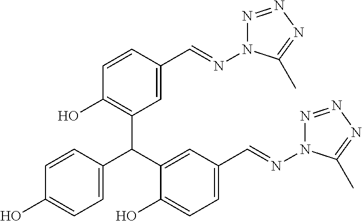 [SMILES: Cc1nnnn1/N=C/c1ccc(O)c(C(c2ccc(O)cc2)c2cc(/C=N/n3nnnc3C)ccc2O)c1] | ViroPharma | | Antimicrob Agents Chemother., June 2005, p. 2460-2466. |
| RFI-641 | 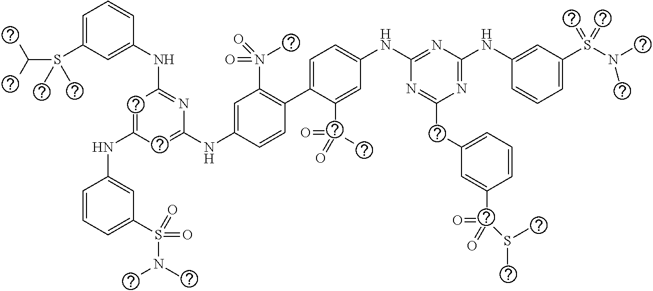 | Wyeth | | Antimicrob Agents Chemother., Mar. 2002, p. 841-847. |
| MBX-300 | 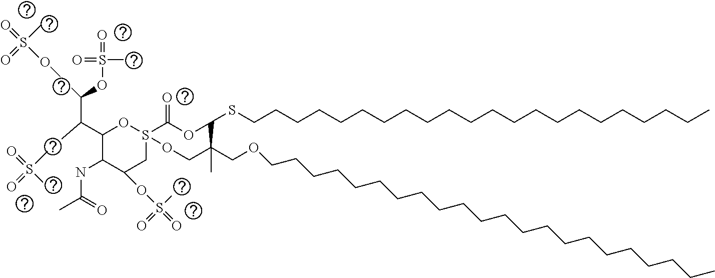 | Microbiotix | | Antiviral Research 61 (2004) 165-171. Alios Biopharma |
| EICAR | 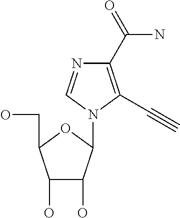 [SMILES: C#Cc1c(C(N)=O)ncn1C1OC(CO)C(O)C1O] | Asahi Kasei Pharma | | Antimicrob Agents Chemother., Feb. 1992, p. 435-439. |
| LY-253963 | 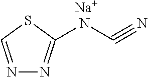 | Eli Lilly | | Antiviral Research 14 (1990) 237-248. |
| RSV-604 | 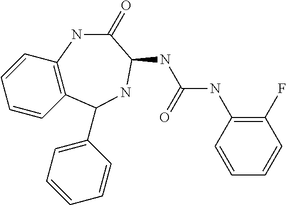 [SMILES: O=C(Nc1ccccc1F)N[C@H]1NC(c2ccccc2)c2ccccc2NC1=O] | Arrow Therapeutics (Novartis) Phase II clinical trials (see US 20130090328) | | Antimicrob Agents Chemother., Sept. 2007, p. 3346-3353. |
| RD3-0028 | | Rational Drug | | |
| | | Design | | |
| | | Laboratories, | | |
| | | JP | | |
| V590 | Antisense RNA | | | |
| BTA-C585 | Small molecule F-protein inhibitor. | | | |
| ALS-8176 | 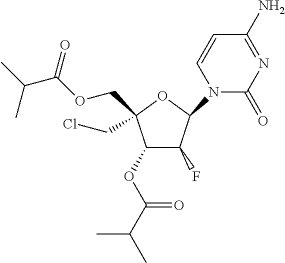 [SMILES: CC(C)C(=O)OC[C@@]1(CCl)O[C@@H](n2ccc(N)nc2=O)[C@@H](F)[C@@H]1OC(=O)C(C)C] | Alios Biopharma | | J. Med. Chem. 2015, 58, 1862-1878 |
| GS-5806 | 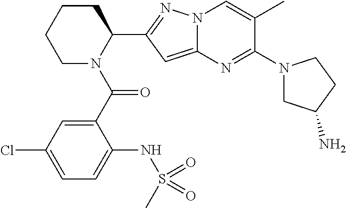 [SMILES: Cc1cn2nc([C@@H]3CCCCN3C(=O)c3cc(Cl)ccc3NS(C)(=O)=O)cc2nc1N1CC[C@H](N)C1] | Gilead; | | J. Med. Chem. 2015, 58, 1630-1643 |
| STP-902 | siRNA | Sirnaomics, | | Listed as STO-92 in |
| | | Inc. | | 14/462937, claim 143 |
| | | | | and also in Emerging |
| | | | | Drugs - Treatment of |
| | | | | Respiratory Syncytial |
| | | | | Virus Infection Past |
| | | | | Present and Future |
| | | | | 2011, Table 3. |
| CL387626 | | Wyeth | | |
| iKT-041 | | Inhibikase | | Listed in 14/462937, |
| | | | | claim 143 and also in |
| | | | | Emerging Drugs - |
| | | | | Treatment of Respiratory |
| | | | | Syncytial Virus |
| | | | | Infection Past |
| | | | | Present and Future |
| | | | | 2011, Table 3. |
| AZ-27 | 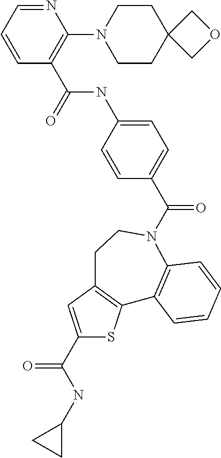 [SMILES: O=C(NC1CC1)c1cc2c(s1)-c1ccccc1N(C(=O)c1ccc(NC(=O)c3cccnc3N3CCC4(CC3)COC4)cc1)CC2] | Astra-Zeneca | | Antimicrobial Agents and Chemotherapy, 2014, 58(7): 3867-3873. |
| CG-100 | | Clarassance, | | |
| | | now called | | |
| | | Therabron | | |
| | | Therapeutics. | | |
| | | Phase II trials | | |
| JMN3-003 | 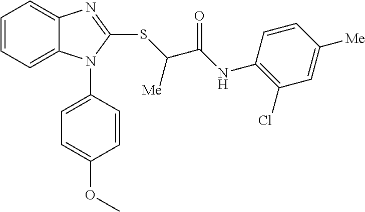 [SMILES: COc1ccc(-n2c(SC(C)C(=O)Nc3ccc(C)cc3Cl)nc3ccccc32)cc1] | Emory University | | PLOS ONE 6(5): e20069. |
| GPAR-3710 | 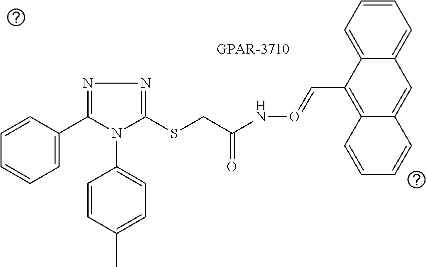 | Georgia State University | | PNAS 2014 E3441-49 |
| Peptide | | Trimeris | | |
| analogues T67, | | | | |
| T118 | | | | |
| Palivizumab | | Medimmune | | |
| (Synagis ®) | | | | |

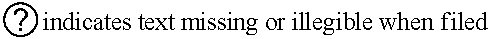

In other embodiments, the invention encompasses a combination comprising the compound of Formula (I) in combination with one or more antimicrobial agents selected from those agents in Table 2, Table 3, and/or Table 4, and also, optionally in combination with one or more additional conventional respiratory treatment agents.

As used herein, the term “conventional respiratory treatment agents” includes any such respiratory infectious disease treatments which treat or alleviate, no matter how slightly, any symptoms arising having a respiratory infectious disease, and are not the compound of Formula (I) or an antimicrobial agent.

For purposes of the present invention, suitable conventional respiratory treatment agents can comprise one or more agents selected from anti-inflammatory agents (e.g., Cox-2 inhibitors, Cox-2/Cox-1 inhibitors, NSAIDs), antihistamines, anticholinergic agents (particularly an M₁/M₂/M₃ receptor antagonist), β₂-adrenoreceptor agonists, steroids (e.g., corticosteroids), PDE4 inhibitor (e.g., Roflumilast), decongestants,

The terms “cyclooxygenase-2 inhibitor”, or “Cox-2 inhibitor”, which can be used interchangeably herein, embrace compounds which inhibit the Cox-2 enzyme regardless of the degree of inhibition of the Cox-1 enzyme, and include pharmaceutically acceptable salts of those compounds. Thus, for purposes of the present invention, a compound is considered a Cox-2 inhibitor irrespective of whether the compound inhibits the Cox-2 enzyme to an equal, greater, or lesser degree than the Cox-1 enzyme. In one embodiment of the present invention, it is preferred that the Cox-2 inhibitor is a non-steroidal anti-inflammatory drug (NSAID). Therefore, preferred materials that can serve as Cox-2 inhibitors of the present invention include non-steroidal anti-inflammatory drug compounds, a pharmaceutically acceptable salt thereof, or a pure (−) or (+) optical isomeric form thereof.

Examples of anti-inflammatory agents include non-steroidal anti-inflammatory drugs (NSAID's). Suitable NSAID compounds that are useful in the present invention include acemetacin, acetyl salicylic acid, alclofenac, alminoprofen, azapropazone, benorylate, benoxaprofen, bucloxic acid, carprofen, choline magnesium trisalicylate, clidanac, clopinac, dapsone, diclofenac, diflunisal, droxicam, etodolac, fenoprofen, fenbufen, fenclofenec, fentiazac, floctafenine, flufenisal, flurbiprofen, (r)-flurbiprofen, (s)-flurbiprofen, furofenac, feprazone, flufenamic acid, fluprofen, ibufenac, ibuprofen, indometacin, indomethacin, indoprofen, isoxepac, isoxicam, ketoprofen, ketorolac, miroprofen, piroxicam, meloxicam, mefenamic, mefenamic acid, meclofenamic acid, meclofen, nabumetone, naproxen, niflumic acid, oxaprozin, oxipinac, oxyphenbutazone, phenylbutazone, podophyllotoxin derivatives, proglumetacin, piprofen, pirprofen, prapoprofen, salicylic acid, salicylate, sudoxicam, suprofen, sulindac, tenoxicam, tiaprofenic acid, tiopinac, tioxaprofen, tolfenamic acid, tolmetin, zidometacin, zomepirac, and 2-fluoro-a-methyl[1,1′-biphenyl]-4-acetic acid, 4-(nitrooxy)butyl ester.

Further suitable NSAID compounds include ibuprofen, naproxen, sulindac, ketoporfen, fenoprofen, tiaprofenic acid, suprofen, etodolac, carprofen, ketrolac, piprofen, indoprofen, salicylic acid, and flurbiprofen.

In one embodiment, the invention encompasses a combination comprising a compound of Formula I, with a β₂-adrenoreceptor agonist.

Examples of β₂-adrenoreceptor agonists include vilanterol, salmeterol (which may be a racemate or a single enantiomer such as the R-enantiomer), salbutamol (which may be a racemate or a single enantiomer such as the R-enantiomer), formoterol (which may be a racemate or a single diastereomer such as the R,R-diastereomer), salmefamol, fenoterol, carmoterol, etanterol, naminterol, clenbuterol, pirbuterol, flerbuterol, reproterol, bambuterol, indacaterol, terbutaline and salts thereof, for example the xinafoate (1-hydroxy-2-naphthalenecarboxylate) salt of salmeterol, the sulphate salt or free base of salbutamol or the fumarate salt of formoterol. In one embodiment the β₂-adrenoreceptor agonists are long-acting β₂-adrenoreceptor agonists, for example, compounds which provide effective bronchodilation for about 12 hours or longer.

Other β₂-adrenoreceptor agonists include those described in WO2002/066422, WO2002/070490, WO2002/076933, WO2003/024439, WO2003/072539, WO2003/091204, WO2004/016578, WO2004/022547, WO2004/037807, WO2004/037773, WO2004/037768, WO2004/039762, WO2004/039766, WO2001/42193 and WO2003/042160.

Further examples of β₂-adrenoreceptor agonists include:

3-(4-{[6-({(2R)-2-hydroxy-2-[4-hydroxy-3-(hydroxymethyl)phenyl]ethyl}amino)hexyl]oxy}butyl) benzenesulfonamide;

3-(3-{[7-({(2R)-2-hydroxy-2-[4-hydroxy-3-hydroxymethyl) phenyl]ethyl}-amino) heptyl]oxy}propyl) benzenesulfonamide;

4-{(1R)-2-[(6-{2-[(2,6-dichlorobenzyl) oxy]ethoxy}hexyl) amino]-1-hydroxyethyl}-2-(hydroxymethyl) phenol;

4-{(1R)-2-[(6-{4-[3-(cyclopentylsulfonyl)phenyl]butoxy}hexyl)amino]-1-hydroxyethyl}-2-(hydroxymethyl)phenol;

N-[2-hydroxyl-5-[(1R)-1-hydroxy-2-[[2-4-[[(2R)-2-hydroxy-2-phenylethyl]amino]phenyl]ethyl]amino]ethyl]phenyl]formamide;

N-2{2-[4-(3-phenyl-4-methoxyphenyl)aminophenyl]ethyl}-2-hydroxy-2-(8-hydroxy-2(1H)-quinolinon-5-yl)ethylamine; and

5-[(R)-2-(2-{4-[4-(2-amino-2-methyl-propoxy)-phenylamino]-phenyl}-ethylamino)-1-hydroxy-ethyl]-8-hydroxy-1H-quinolin-2-one.

The β₂-adrenoreceptor agonist may be in the form of a salt formed with a pharmaceutically acceptable acid selected from sulphuric, hydrochloric, fumaric, hydroxynaphthoic (for example 1- or 3-hydroxy-2-naphthoic), cinnamic, substituted cinnamic, triphenylacetic, sulphamic, sulphanilic, naphthaleneacrylic, benzoic, 4-methoxybenzoic, 2- or 4-hydroxybenzoic, 4-chlorobenzoic and 4-phenylbenzoic acid.

Suitable anti-inflammatory agents include corticosteroids. Examples of corticosteroids which may be used in combination with the compound Formula I of the invention are those oral and inhaled corticosteroids and their pro-drugs which have anti-inflammatory activity. Examples include methyl prednisolone, prednisolone, dexamethasone, fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate), 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, 6α,9α-difluoro-11β-hydroxy-16α-methyl-3-oxo-17α-(2,2,3,3-tetramethycyclopropylcarbonyl)oxy-androsta-1,4-diene-17β-carboxylic acid cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester, beclomethasone esters (for example the 17-propionate ester or the 17,21-dipropionate ester), budesonide, flunisolide, mometasone esters (for example mometasone furoate), triamcinolone acetonide, rofleponide, ciclesonide (16α,17-[[(R)-cyclohexylmethylene]bis(oxy)]-11β,21-dihydroxy-pregna-1,4-diene-3,20-dione), butixocort propionate, RPR-106541, and ST-126. In one embodiment corticosteroids include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-(2,2,3,3-tetramethycyclopropylcarbonyl)oxy-androsta-1,4-diene-17β-carboxylic acid cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester. In one embodiment the corticosteroid is 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Examples of corticosteroids may include those described in WO2002/088167, WO2002/100879, WO2002/12265, WO2002/12266, WO2005/005451, WO2005/005452, WO2006/072599 and WO2006/072600.

Non-steroidal compounds having glucocorticoid agonism that may possess selectivity for transrepression over transactivation and that may be useful in combination therapy include those covered in the following published patent applications and patents: WO1998/54159, WO2000/66590, WO2001/16128, WO2002/02565,

WO2003/059899, WO2003/061651, WO2003/082280, WO2003/082787, WO2003/082827, WO2003/086294, WO2003/101932, WO2003/104195,

WO2004/005229, WO2004/009017, WO2004/018429, WO2004/026248, WO2006/000398, WO2006/000401, WO2006/015870, WO2006/108699, WO2007/000334, WO2007/054294, WO2007/122165, WO2007/144327 and WO2008/000777.

In one embodiment the invention provides the use of the compounds of formula (I) in combination with a phosphodiesterase 4 (PDE4) inhibitor, for example in the case of a formulation adapted for inhalation. The PDE4 inhibitor useful in this aspect of the invention may be any compound that is known to or which is discovered to act as a PDE4 inhibitor, e.g. as an inhibitor of PDE4B and/or PDE4D.

PDE4 inhibitory compounds include cis-4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]. Also, cis-4-cyano-4-[3-(cyclopentyloxy)-4-methoxyphenyl]cyclohexane-1-carboxylic acid (also known as cilomilast) and its salts, esters, pro-drugs or physical forms, which is described in U.S. Pat. No. 5,552,438 issued 3 Sep. 1996; this patent and the compounds it discloses are incorporated herein in full by reference.

Other PDE4 inhibitory compounds include AWD-12-281 (N-(3,5-dichloro-4-pyridinyl)-1-[4-fluorophenyl)methyl]-5-hydroxy-α-oxo-1H-indol-3-acetamide) from Elbion (Hofgen, N. et al. 15th EFMC Int Symp Med Chem (September 6-10, Edinburgh) 1998, Abst P.98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998]1998, 12 (Suppl. 28): Abst P2393); roflumilast (3-(cyclopropylmethoxy)-N-(3,5-dichloro-4-pyridinyl)-4-(difluoromethoxy)benzamide) (see EP 0 706 513 B1 to Byk Gulden Lomberg, e.g. see Example 5 thereof); a phthalazinone (WO1999/47505) from Byk-Gulden; Pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther,1998, 284(1): 162), and T2585.

Further PDE4 inhibitory compounds are disclosed in the published international patent applications WO2004/024728, WO2004/056823, WO2004/103998 (e.g. Example 399 or 544 disclosed therein), WO2005/058892, WO2005/090348, WO2005/090353, and WO2005/090354, all in the name of Glaxo Group Limited.

Examples of anticholinergic agents are those compounds that act as antagonists at the muscarinic receptors, in particular those compounds which are antagonists of the M₁ or M₃ receptors, dual antagonists of the M₁/M₃ or M₂/M₃, receptors or pan-antagonists of the M₁/M₂/M₃ receptors. Exemplary compounds for administration via inhalation include ipratropium (for example, as the bromide, CAS 22254-24-6, sold under the name Atrovent), oxitropium (for example, as the bromide, CAS 30286-75-0) and tiotropium (for example, as the bromide, CAS 136310-93-5, sold under the name Spiriva). Also of interest are revatropate (for example, as the hydrobromide, CAS 262586-79-8) and LAS-34273 which is disclosed in WO2001/04118. Exemplary compounds for oral administration include pirenzepine (CAS 28797-61-7), darifenacin (CAS 133099-04-4, or CAS 133099-07-7 for the hydrobromide sold under the name Enablex), oxybutynin (CAS 5633-20-5, sold under the name Ditropan), terodiline (CAS 15793-40-5), tolterodine (CAS 124937-51-5, or CAS 124937-52-6 for the tartrate, sold under the name Detrol), otilonium (for example, as the bromide, CAS 26095-59-0, sold under the name Spasmomen), trospium chloride (CAS 10405-02-4) and solifenacin (CAS 242478-37-1, or CAS 242478-38-2 for the succinate also known as YM-905 and sold under the name Vesicare).

Additional compounds are disclosed in WO 2005/037280, WO 2005/046586 and WO 2005/104745, incorporated herein by reference. The present combinations include, but are not limited to:

(3-endo)-3-(2,2-di-2-thienylethenyl)-8,8-dimethyl-8-azoniabicyclo[3.2.1]octane iodide;

(3-endo)-3-(2-cyano-2,2-diphenylethyl)-8,8-dimethyl-8-azoniabicyclo[3.2.1]octane bromide;

4-[hydroxy(diphenyl)methyl]-1-{2-[(phenylmethyl)oxy]ethyl}-1-azoniabicyclo[2.2.2]octane bromide; and

(1R,5S)-3-(2-cyano-2,2-diphenylethyl)-8-methyl-8-{2-[(phenylmethyl)oxy]ethyl}-8-azoniabicyclo[3.2.1]octane bromide.

In one embodiment the invention provides a combination comprising the compound of Formula (I) or a pharmaceutically acceptable salt thereof together with an antihistamine, such as an H1 antagonist. Examples of suitable H1 antagonists include, without limitation, diphenhydramine, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine and triprolidine, particularly azelastine, cetirizine, levocetirizine, efletirizine and fexofenadine.

In a another embodiment the invention provides a combination comprising the compound of Formula (I), or a pharmaceutically acceptable salt thereof together with an H3 antagonist (and/or inverse agonist). Examples of H3 antagonists include, for example, those compounds disclosed in WO2004/035556, WO2006/045416, WO2006/090142, WO2006/125665, WO2007/009739 and WO2007/009741. In a another embodiment the invention provides a combination comprising the compound of Formula (I), or a pharmaceutically acceptable salt thereof together with an H1/H3 dual antagonist (and/or inverse agonist). Examples of H1/H3 dual antagonists include, for example, those compounds disclosed in WO2004/035556, WO2007/071691, WO2007/122156 and WO2007/135081. In a further embodiment the invention provides a combination comprising the compound of Formula (I), or a pharmaceutically acceptable salt thereof together with an H1/H3 dual antagonist selected from 3-(4-{[4-(4-{([3-(3,3-dimethyl-1-piperidinyl)propyl]oxy}phenyl)-1-piperidinyl]carbonyl}-1-naphthalenyl) propanoic acid and 4-[(4-chlorophenyl)methyl]-2-({(2R)-1-[4-(4-{[3-(hexahydro-1H-azepin-1-yl)propyl]oxy}phenyl)butyl]-2-pyrrolidinyl}methyl)-1(2H)-phthalazinone. Other histamine receptor antagonists which may be used in combination with the compounds of the present invention include antagonists (and/or inverse agonists) of the H4 receptor, for example, the compounds disclosed in Jablonowski et al., J. Med. Chem. 46:3957-3960 (2003).

Additional suitable conventional respiratory treatment agents include sodium cromoglycate, nedocromil sodium, phosphodiesterase (PDE) inhibitors (for example, theophylline, PDE4 inhibitors or mixed PDE3/PDE4 inhibitors), leukotriene antagonists, inhibitors of leukotriene synthesis (for example montelukast), iNOS inhibitors, tryptase and elastase inhibitors, beta-2 integrin antagonists and adenosine receptor agonists or antagonists (e.g. adenosine 2a agonists), cytokine antagonists (for example chemokine antagonists, such as a CCR3 antagonist) or inhibitors of cytokine synthesis, or 5-lipoxygenase inhibitors. In one embodiment, the invention encompasses iNOS (inducible nitric oxide synthase) inhibitors for oral administration. Examples of iNOS inhibitors include those disclosed in WO1993/13055, WO1998/30537, WO2002/50021, WO1995/34534 and WO1999/62875. Examples of CCR3 inhibitors include those disclosed in WO2002/26722.

In other embodiments of the present invention, the conventional respiratory treatment agents may be selected from the group consisting of fenamates, pyrrolealkanoic acids, pyrazolone derivatives, oxicams, pramoxine, azatadine, meclizine, promethazine bromodiphenhydramine, brompheniramine, brompheniramine maleate, carbinoxamine, chlorpheniramine, dexchlorpheniramine, diphenhydramine, doxylamine, phenindamine, pheniramine, phenyltoloxamine, pyrilamine, triprolidine, clemastine, dimenhydrinate, cetirizine, terfenadine, astemizole, loratadine, acrivastine, hydroxyzine, meclozine, compazine, imipramine, doxopin, amitryptoline, tripelennamine, fexofenadine, azatadine, ephedrine, ephinephrine, levodesoxyephedrine, oxymetazoline, naphazoline, phenylephrine, phenylpropanolamine, propylhexedrine, pseudoephedrine, xylometazoline, chlorhexidine, mercurochrome, povidone iodine, polyhyroxine iodine, cresylate, hydrocortisone, prednisone, fluprednisolone, dexamethasone, betamethasone, betamethasone valerate, methylprednisolone, fluocinolone acetonide, flurandrenolone acetonide, fluorometholone, cortisone, prednisolone, alclometasone, amcinonide, betamethasone, clobetasol, clocortolone, desonide, desoximetasone, diflorasone, fluocinonide, flurandrenolide, fluticasone, halcinonide, halobetasol, mometasone, flumethasone, prednicarbate, triamcinolone, clotrimazole, griseofulvin, undecylenic, econazole, miconazole, ketaconazole, sulconazole, oxiconazole, fluconazole, itraconazole, nystatin, naftifine, terbinafine, ciclopirox, butenafine, haloprogin, tolnaftate, tobramycin plus dexamethasone, m-cresyl acetate, bis-(2-pyridyl-1-oxide) disulfide, acetaminophen, mafenide, and mixtures thereof.

Thus, in one embodiment of the present invention, there is provided a composition comprising danirixin in combination with a neuraminidase inhibitor compound.

In another embodiment of the present invention, there is provided a composition comprising danirixin in combination with zanamivir.

In another embodiment of the present invention, there is provided a composition comprising danirixin in combination with oseltamivir.

In another embodiment of the present invention, there is provided a composition comprising danirixin in combination with ribavirin.

In another embodiment of the present invention, there is provided a composition comprising danirixin in combination with favipiravir.

In another embodiment of the present invention, there is provided a composition comprising danirixin in combination with one or more antimicrobial agents selected from Table 4.

In another embodiment of the present invention, there is provided a pharmaceutical composition comprising danirixin in combination with a neuraminidase inhibitor compound and a pharmaceutically acceptable excipient.

In another embodiment of the present invention, there is provided a pharmaceutical composition comprising danirixin in combination with zanamivir and a pharmaceutically acceptable excipient.

In another embodiment of the present invention, there is provided a pharmaceutical composition comprising danirixin in combination with oseltamivir and a pharmaceutically acceptable excipient.

In another embodiment of the present invention, there is provided a pharmaceutical composition comprising danirixin in combination with ribavirin and a pharmaceutically acceptable excipient.

In another embodiment of the present invention, there is provided a pharmaceutical composition comprising danirixin in combination with one or more antimicrobial agents selected from Table 4 and a pharmaceutically acceptable excipient.

In another embodiment of the present invention, there is provided a method for treating a respiratory infectious disease in a subject, the method comprising administering to a subject suffering from a respiratory infectious disease danirixin in combination with a neuraminidase inhibitor compound.

In another embodiment of the present invention, there is provided a method for treating a respiratory infectious disease in a subject, the method comprising administering to a subject suffering from a respiratory infectious disease danirixin in combination with zanamivir.

In another embodiment of the present invention, there is provided a method for treating a respiratory infectious disease in a subject, the method comprising administering to a subject suffering from a respiratory infectious disease danirixin in combination with oseltamivir.

In another embodiment of the present invention, there is provided a method for treating a respiratory infectious disease in a subject, the method comprising administering to a subject suffering from a respiratory infectious disease danirixin in combination with ribavirin.

In another embodiment of the present invention, there is provided a method for treating a respiratory infectious disease in a subject, the method comprising administering to a subject suffering from a respiratory infectious disease danirixin in combination with favipiravir.

In another embodiment of the present invention, there is provided a method for treating a respiratory infectious disease in a subject, the method comprising administering to a subject suffering from a respiratory infectious disease danirixin in combination with one or more antimicrobial agents selected from Table 4.

In another embodiment of the present invention, there is provided a method for treating a respiratory infectious disease in a subject, the method comprising administering to a subject suffering from a respiratory infectious disease danirixin in combination with a neuraminidase inhibitor compound, wherein the respiratory infectious disease is influenza.

In another embodiment of the present invention, there is provided a method for treating a respiratory infectious disease in a subject, the method comprising administering to a subject suffering from a respiratory infectious disease danirixin in combination with a neuraminidase inhibitor compound, wherein the combination of danirixin and neuraminidase inhibitor compound are administered in the same dosage form.

In another embodiment of the present invention, there is provided a method for treating a respiratory infectious disease in a subject, the method comprising administering to a subject suffering from a respiratory infectious disease danirixin in combination with a neuraminidase inhibitor compound, wherein the combination of danirixin and neuraminidase inhibitor compound are administered simultaneously.

In another embodiment of the present invention, there is provided a method for treating a respiratory infectious disease in a subject, the method comprising administering to a subject suffering from a respiratory infectious disease danirixin in combination with a neuraminidase inhibitor compound, wherein the combination of danirixin and neuraminidase inhibitor compound are administered separately.

In another embodiment of the present invention, there is provided a method for treating a respiratory infectious disease in a subject, the method comprising administering to a subject suffering from a respiratory infectious disease danirixin in combination with a neuraminidase inhibitor compound, wherein the combination of danirixin and neuraminidase inhibitor compound are administered in the same dosage form.

In another embodiment of the present invention, there is provided a method for treating a respiratory infectious disease in a subject, the method comprising administering to a subject suffering from a respiratory infectious disease danirixin in combination with a neuraminidase inhibitor compound, wherein the combination of the compound of danirixin and neuraminidase inhibitor compound are administered simultaneously.

In another embodiment of the present invention, there is provided a method for treating a respiratory infectious disease in a subject, the method comprising administering to a subject suffering from a respiratory infectious disease danirixin in combination with a ribavirin.

In another embodiment of the present invention, there is provided a method for treating a respiratory infectious disease in a subject, the method comprising administering to a subject suffering from a respiratory infectious disease danirixin in combination with a ribavirin, wherein the combination of the compound of danirixin and ribavirin are administered simultaneously.

In another embodiment of the present invention, there is provided a method for treating a respiratory infectious disease in a subject, the method comprising administering to a subject suffering from a respiratory infectious disease danirixin in combination with a ribavirin, wherein the combination of danirixin and ribavirin are administered in the same dosage form.

In another embodiment of the present invention, there is provided a method for treating a respiratory infectious disease in a subject, the method comprising administering to a subject suffering from a respiratory infectious disease danirixin in combination with a ribavirin, wherein the combination of the compound of danirixin and ribavirin are administered simultaneously.

In another embodiment of the present invention, there is provided a method for treating a respiratory infectious disease in a subject, the method comprising administering to a subject suffering from a respiratory infectious disease danirixin in combination with a ribavirin, wherein the combination of danirixin and ribavirin are administered separately.

A method for treating influenza in a subject, the method comprising administering danirixin to a subject suffering from influenza.

A method for treating RSV in a subject, the method comprising administering danirixin to a subject suffering from RSV.

A pharmaceutical composition for intravenous administration comprising: danirixin as a hydrobromide salt in aqueous solution.

A pharmaceutical composition for intravenous administration comprising: danirixin as a hydrobromide salt and a pharmaceutically acceptable excipient in aqueous solution.

A pharmaceutical composition for intravenous administration comprising: danirixin as a hydrobromide salt and a pharmaceutically acceptable excipient in aqueous solution, wherein the pharmaceutically acceptable excipient comprises cyclodextrin.

A pharmaceutical composition for intravenous administration comprising: danirixin as a hydrobromide salt and a pharmaceutically acceptable excipient in aqueous solution, wherein the pharmaceutically acceptable excipient comprises β-cyclodextrin.

A pharmaceutical composition for intravenous administration comprising: danirixin as a hydrobromide salt and a pharmaceutically acceptable excipient in aqueous solution, wherein the pharmaceutically acceptable excipient comprises sulfobutylether.

A pharmaceutical composition for intravenous administration comprising: danirixin as a hydrobromide salt and a pharmaceutically acceptable excipient in aqueous solution, wherein the pharmaceutically acceptable excipient comprises β-cyclodextrin and sulfobutylether.

A pharmaceutical composition for intravenous administration comprising: danirixin as a hydrobromide salt and a pharmaceutically acceptable excipient in aqueous solution, wherein the pharmaceutically acceptable excipient comprises Captisol®.

A method for treating a respiratory infectious disease in a subject, the method comprising administering a pharmaceutical composition for intravenous administration comprising: danirixin as a hydrobromide salt and a pharmaceutically acceptable excipient in aqueous solution, to a subject suffering from a respiratory infectious disease.

A method for treating a respiratory infectious disease in a subject, the method comprising administering a pharmaceutical composition for intravenous administration comprising: danirixin as a hydrobromide salt and a pharmaceutically acceptable excipient in aqueous solution, to a subject suffering from a respiratory infectious disease, wherein the pharmaceutically acceptable excipient comprises cyclodextrin.

A method for treating a respiratory infectious disease in a subject, the method comprising administering a pharmaceutical composition for intravenous administration comprising: danirixin as a hydrobromide salt and a pharmaceutically acceptable excipient in aqueous solution, to a subject suffering from a respiratory infectious disease, wherein the pharmaceutically acceptable excipient comprises β-cyclodextrin.

### Administration and Formulation

In another embodiment, there is provided a pharmaceutical composition comprising a pharmaceutically acceptable diluent and a therapeutically effective amount of the compound of Formula (I), or a pharmaceutically acceptable salt thereof, alone or in combination with an antimicrobial agent, and/or a conventional respiratory treatment agent.

The compounds of the present invention can be supplied in the form of a pharmaceutically acceptable salt. The terms “pharmaceutically acceptable salt” refer to salts prepared from pharmaceutically acceptable inorganic and organic acids and bases. Accordingly, the word “or” in the context of “a compound or a pharmaceutically acceptable salt thereof” is understood to refer to either a compound or a pharmaceutically acceptable salt thereof (alternative), or a compound and a pharmaceutically acceptable salt thereof (in combination).

As used herein, the term “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication. The skilled artisan will appreciate that pharmaceutically acceptable salts of compounds according to Formulas I, II, or III may be prepared. These pharmaceutically acceptable salts may be prepared in situ during the final isolation and purification of the compound, or by separately reacting the purified compound in its free acid or free base form with a suitable base or acid, respectively.

Illustrative pharmaceutically acceptable acid salts of the compounds of the present invention can be prepared from the following acids, including, without limitation formic, acetic, propionic, benzoic, succinic, glycolic, gluconic, lactic, maleic, malic, tartaric, citric, nitic, ascorbic, glucuronic, maleic, fumaric, pyruvic, aspartic, glutamic, benzoic, hydrochloric, hydrobromic, hydroiodic, isocitric, trifluoroacetic, pamoic, propionic, anthranilic, mesylic, oxalacetic, oleic, stearic, salicylic, p-hydroxybenzoic, nicotinic, phenylacetic, mandelic, embonic (pamoic), methanesulfonic, phosphoric, phosphonic, ethanesulfonic, benzenesulfonic, pantothenic, toluenesulfonic, 2-hydroxyethanesulfonic, sulfanilic, sulfuric, salicylic, cyclohexylaminosulfonic, algenic, β-hydroxybutyric, galactaric and galacturonic acids. Preferred pharmaceutically acceptable salts include the salts of hydrochloric acid and trifluoroacetic acid.

Illustrative pharmaceutically acceptable inorganic base salts of the compounds of the present invention include metallic ions. More preferred metallic ions include, but are not limited to, appropriate alkali metal salts, alkaline earth metal salts and other physiological acceptable metal ions. Salts derived from inorganic bases include aluminum, ammonium, calcium, copper, ferric, ferrous, lithium, magnesium, manganic salts, manganous, potassium, sodium, zinc, and the like and in their usual valences. Exemplary base salts include aluminum, calcium, lithium, magnesium, potassium, sodium and zinc. Other exemplary base salts include the ammonium, calcium, magnesium, potassium, and sodium salts. Still other exemplary base salts include, for example, hydroxides, carbonates, hydrides, and alkoxides including NaOH, KOH, Na₂CO₃, K₂CO₃, NaH, and potassium-t-butoxide.

Salts derived from pharmaceutically acceptable organic non-toxic bases include salts of primary, secondary, and tertiary amines, including in part, trimethylamine, diethylamine, N, N′-dibenzylethylenediamine, chloroprocaine, choline, diethanolamine, ethylenediamine, meglumine (N-methylglucamine) and procaine; substituted amines including naturally occurring substituted amines; cyclic amines; quaternary ammonium cations; and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

All of the above salts can be prepared by those skilled in the art by conventional means from the corresponding compound of the present invention. For example, the pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. The salt may precipitate from solution and be collected by filtration or may be recovered by evaporation of the solvent. The degree of ionisation in the salt may vary from completely ionised to almost non-ionised. Lists of suitable salts are found in Remington's Pharmaceutical Sciences, 17th ed., Mack Publishing Company, Easton, Pa., 1985, p.1418, the disclosure of which is hereby incorporated by reference only with regards to the lists of suitable salts.

The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water. Pharmaceutically acceptable solvates include hydrates and other solvates wherein the solvent of crystallization may be isotopically substituted, e.g. D₂O, d₆-acetone, d₆-DMSO.

The compound of Formula (I) containing one or more asymmetric carbon atoms can exist as two or more stereoisomers. Where the compound of Formula (I) (or antimicrobial agentand/or conventional respiratory treatment agent) contains an alkenyl or alkenylene group or a cycloalkyl group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

Included within the scope of the present invention are all stereoisomers, geometric isomers and tautomeric forms of the compounds of Formula (I) (or antimicrobial agentand/or conventional respiratory treatment agent), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art. [see, for example, “Stereochemistry of Organic Compounds” by E L Eliel (Wiley, N.Y., 1994).

The present invention includes all pharmaceutically acceptable isotopically-labelled compounds, wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of the present invention are embraced, including, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Isotopically-labelled compounds can generally be prepared by conventional techniques known to those skilled in the art or by processes analogous to those described herein using an appropriate isotopically-labelled reagents in place of the non-labelled reagent previously employed.

The compounds of the present invention may be administered as prodrugs. Thus, certain derivatives of the compounds of the present invention, which may have little or no pharmacological activity themselves can, when administered into or onto the body, be converted into compounds having the desired activity, for example, by hydrolytic cleavage. Such derivatives are referred to as ‘prodrugs’.

The compositions of the present invention are comprised of, in general, at least one chemical entity described herein in combination with at least one pharmaceutically acceptable excipient. Acceptable excipients are non-toxic, aid administration, and do not adversely affect the therapeutic benefit of the at least one chemical entity described herein. Such excipient may be any solid, liquid, semi-solid or, in the case of an aerosol composition, gaseous excipient that is generally available to one of skill in the art.

Compounds herein, pharmaceutically acceptable salts thereof and pharmaceutical compositions incorporating such may conveniently be administered by any of the routes conventionally used for drug administration. The compounds of herein may be administered in conventional dosage forms prepared by combining the compound of Formula (I) with standard pharmaceutical carriers according to conventional procedures. The compounds herein may also be administered in conventional dosages in combination with a known, second therapeutically active compound.

Administration of the chemical entities described herein can be via any of the accepted modes of administration for agents that serve similar utilities including, but not limited to, orally, systemic (e.g., transdermal, intranasal or by suppository), or parenteral (e.g., intramuscular, intravenous or subcutaneous), sublingually, topically, intraperitoneally, intrapulmonarilly, vaginally, rectally, or intraocularly. In some embodiments, the compound of Formula (I) is orally parenteral administered. In other embodiments, the compound of Formula (I) is administered by an intrapulmonary route. In still other embodiments, the antimicrobial agent is administered by an intrapulmonary route.

In still further embodiments, the compound of Formula (I) is administered intravenously. In one embodiment, the compound of Formula (I) is administered intravenously as a solution containing from 0.1 to 10 mg/mL of the compound of Formula (I) as a free base in water for injection and comprising β-cyclodextrin and sulfobutylether. In another embodiment, the compound of Formula (I) is administered intravenously as a solution containing 2 mg/mL of the compound of Formula (I) as a free base in water for injection and comprising β-cyclodextrin and sulfobutylether. In other embodiments, the compound of Formula (I) is administered intravenously as a solution containing 2 mg/mL of the compound of Formula (I) as a free base in water for injection and comprising β-cyclodextrin and sulfobutylether, and wherein each vial of the intravenous solution of the compound of Formula (I) contains 13 mL of 2 mg/mL of the compound of Formula (I).

Pharmaceutical compositions or formulations include solid, semi-solid, liquid and aerosol dosage forms, such as, e.g., tablets, capsules, powders, liquids, suspensions, suppositories, aerosols or the like. The chemical entities can also be administered in sustained or controlled release dosage forms, including depot injections, osmotic active pumps, pills, transdermal (including electrotransport) patches, and the like, for prolonged and/or timed, pulsed administration at a predetermined rate. In certain embodiments, the compositions are provided in unit dosage forms suitable for single administration of a precise dose.

The chemical entities described herein can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrin, cyclodextrine, cyclodextrin derivatives and cyclodextrine derivatives, sorbitan monolaurate, triethanolamine acetate, triethanolamine oleate). Generally, depending on the intended mode of administration, the pharmaceutical composition will contain about 0.005% to 95%; in certain embodiments, about 0.5% to 50% by weight of a chemical entity. Actual methods of preparing such dosage forms are known, or will be apparent, to those skilled in this art; for example, see Remington's Pharmaceutical Sciences, Mack Publishing Company, Easton, Pa.

In certain embodiments, the compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule.

Liquid pharmaceutically administrable compositions can, for example, be prepared by dissolving, dispersing, etc. at least one chemical entity and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of chemical entities contained in such parenteral compositions is highly dependent on the specific nature thereof, as well as the activity of the chemical entities and the needs of the subject. However, percentages of active ingredient of 0.01% to 10% in solution are employable, and will be higher if the composition is a solid which will be subsequently diluted to the above percentages. In certain embodiments, the composition will comprise from about 0.2 to 2% of the active agent in solution.

Pharmaceutical compositions of the chemical entities described herein may also be administered to the respiratory tract as an aerosol or solution for a nebulizer, or as a microfine powder for insufflation, alone or in combination with an inert carrier such as lactose. In such a case, the particles of the pharmaceutical composition have diameters of less than 50 microns, in certain embodiments, less than 10 microns.

These procedures may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof.

The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time delay material well known to the art, such as glyceryl monostearate or glyceryl distearate alone or with a wax. A wide variety of pharmaceutical forms can be employed. Thus, if a solid carrier is used, the preparation can be tableted, placed in a hard gelatin capsule in powder or pellet form or in the form of a troche or lozenge. The amount of solid carrier will vary widely but preferably will be from about 25 mg. to about 1 g. When a liquid carrier is used, the preparation will be in the form of a syrup, emulsion, soft gelatin capsule, sterile injectable liquid such as an ampoule or non-aqueous liquid suspension.

In general, the chemical entities provided will be administered in a therapeutically effective amount by any of the accepted modes of administration for agents that serve similar utilities. The actual amount of the chemical entity, i.e., the active ingredient, will depend upon numerous factors such as the severity of the disease to be treated, the age and relative health of the subject, the potency of the chemical entity used, the route and form of administration, and other factors. The drug can be administered more than once a day, such as once or twice a day.

Therapeutically effective amounts of the chemical entities described herein may range from approximately 0.01 to 200 mg per kilogram body weight of the recipient per day; such as about 0.01-100 mg/kg/day, for example, from about 0.1 to 50 mg/kg/day. Thus, for administration to a 70 kg person, the dosage range may be about 1-2000 mg per day.

Another manner for administering the provided chemical entities is inhalation. The choice of formulation depends on various factors such as the mode of drug administration and bioavailability of the drug substance. For delivery via inhalation the chemical entity can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDIs typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Compounds herein may be administered topically, that is by non-systemic administration. This includes the application of the compound of Formula (I) externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration. Formulations suitable for topical administration include liquid or semi-liquid preparations suitable for penetration through the skin to the site of inflammation such as liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as stearic or oleic acid together with an alcohol such as propylene glycol or a macro gel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, 15 and other ingredients such as lanolin, may also be included.

Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

The compounds described herein may also be administered by inhalation, that is by intranasal and oral inhalation administration. Appropriate dosage forms for such administration, such as an aerosol formulation or a metered dose inhaler, may be prepared by conventional techniques. In one embodiment of the present invention, the agents of the present invention are delivered via oral inhalation or intranasal administration. Appropriate dosage forms for such administration, such as an aerosol formulation or a metered dose inhaler, may be prepared by conventional techniques.

For administration by inhalation the compounds may be delivered in the form of an aerosol spray presentation from pressurized packs or a nebulizer, with the use of a suitable propellant, e.g. dichlorodifluoromethane, trichlorofluoromethane, dichlorotetrafluoroethane, a hydrofluoroalkane such as tetrafluoroethane or heptafluoropropane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of a compound of the invention and a suitable powder base such as lactose or starch.

Dry powder compositions for topical delivery to the lung by inhalation may, for example, be presented in capsules and cartridges of for example gelatin or blisters of for example laminated aluminum foil, for use in an inhaler or insufflator. Powder blend formulations generally contain a powder mix for inhalation of the compound of the invention and a suitable powder base (carrier/diluent/excipient substance) such as mono-, di or poly-saccharides (e.g. lactose or starch). Use of lactose is preferred.

Each capsule or cartridge may generally contain between 20 μg-1000 mg of the compound of Formula (I) optionally in combination with another therapeutically active ingredient, such as an antimicrobial agent. Alternatively, the compound of the invention may be presented without excipients. Suitably, the packing/medicament dispenser is of a type selected from the group consisting of a reservoir dry powder inhaler (RDPI), a multi-dose dry powder inhaler (MDPI), and a metered dose inhaler (MDI). By reservoir dry powder inhaler (RDPI) it is meant an inhaler having a reservoir form pack suitable for comprising multiple (un-metered doses) of medicament in dry powder form and including means for metering medicament dose from the reservoir to a delivery position. The metering means may for example comprise a metering cup, which is movable from a first position where the cup may be filled with medicament from the reservoir to a second position where the metered medicament dose is made available to the patient for inhalation. By multi-dose dry powder inhaler (MDPI) is meant an inhaler suitable for dispensing medicament in dry powder form, wherein the medicament is comprised within a multi-dose pack containing (or otherwise carrying) multiple, define doses (or parts thereof) of medicament. In a preferred aspect, the carrier has a blister pack form, but it could also, for example, comprise a capsule-based pack form or a carrier onto which medicament has been applied by any suitable process including printing, painting and vacuum occlusion.

In the case of multi-dose delivery, the formulation can be pre-metered (e.g. as in Diskus, see U.S. Pat. Nos. 6,632,666, 5,860,419, 5,873,360 5,622,166 and 5,590,645 or Diskhaler, see, U.S. Pat. Nos. 4,627,432, 4,778,054, 4,811,731, 5,035,237, the disclosures of which are hereby incorporated by reference) or metered in use (e. g. as in Turbuhaler, see U.S. Pat. No. 4,524,769 or in the devices described in U.S. Pat. No. 6,321,747 the disclosures of which are hereby incorporated by reference). An example of a unit-dose device is Rotahaler (see U.S. Pat. Nos. 4,353,656 and 5,724,959, the disclosures of which are hereby incorporated by reference).

The Diskus inhalation device comprises an elongate strip formed from a base sheet having a plurality of recesses spaced along its length and a lid sheet hermetically but peelably sealed thereto to define a plurality of containers, each container having therein an inhalable formulation containing the compound of Formula (I) preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet. In one aspect, the multi-dose pack is a blister pack comprising multiple blisters for containment of medicament in dry powder form. The blisters are typically arranged in regular fashion for ease of release of medicament there from. In one aspect, the multi-dose blister pack comprises plural blisters arranged in generally circular fashion on a disc-form blister pack. In another aspect, the multidose blister pack is elongate in form, for example comprising a strip or a tape. In one aspect, the multi-dose blister pack is defined between two members peelably secured to one another. U.S. Pat. Nos. 5,860,419, 5,873,360 and 5,590,645 describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary. Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable. The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined there between and such that during movement between is nondispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085. Additionally, intra-nasal delivery of the present compounds is effective.

To formulate an effective pharmaceutical nasal composition, the medicament must be delivered readily to all portions of the nasal cavities (the target tissues) where it performs its pharmacological function. Additionally, the medicament should remain in contact with the target tissues for relatively long periods of time. The longer the medicament remains in contact with the target tissues, the medicament must be capable of resisting those forces in the nasal passages that function to remove particles from the nose. Such forces, referred to as ‘mucociliary clearance’, are recognized as being extremely effective in removing particles from the nose in a rapid manner, for example, within 10-30 minutes from the time the particles enter the nose.

Other desired characteristics of a nasal composition are that it must not contain ingredients which cause the user discomfort, that it has satisfactory stability and shelf-life properties, and that it does not include constituents that are considered to be detrimental to the environment, for example ozone depletors. A suitable dosing regime for the formulation of the present invention when administered to the nose would be for the patient to inhale deeply subsequent to the nasal cavity being cleared. During inhalation, the formulation would be applied to one nostril while the other is manually compressed. This procedure would then be repeated for the other nostril. One means for applying the formulation of the present invention to the nasal passages is by use of a pre-compression pump. Most preferably, the pre-compression pump will be a VP7 model manufactured by Valois SA. Such a pump is beneficial as it will ensure that the formulation is not released until a sufficient force has been applied, otherwise smaller doses may be applied. Another advantage of the precompression pump is that atomization of the spray is ensured as it will not release the formulation until the threshold pressure for effectively atomizing the spray has been achieved. Typically, the VP7 model may be used with a bottle capable of holding 10-50 ml of a formulation. Each spray will typically deliver 50-100 μl of such a formulation; therefore, the VP7 model is capable of providing at least 100 metered doses.

Spray compositions for topical delivery to the lung by inhalation may for example be formulated as aqueous solutions or suspensions or as aerosols delivered from pressurized packs, such as a metered dose inhaler, with the use of a suitable liquefied propellant. Aerosol compositions suitable for inhalation can be either a suspension or a solution and generally contain the compound of Formula I, optionally in combination with another therapeutically active ingredient and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetra-fluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants, e.g., oleic acid or lecithin and cosolvents, e.g. ethanol. Pressurized formulations will generally be retained in a canister (e.g. an aluminum canister) closed with a valve (e.g. a metering valve) and fitted into an actuator provided with a mouthpiece. Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. To achieve these particle sizes the particles of the active ingredient as produced may be size reduced by conventional means e.g., by micronization. The desired fraction may be separated out by air classification or sieving. Suitably, the particles will be crystalline in form. When an excipient such as lactose is employed, generally, the particle size of the excipient will be much greater than the inhaled medicament within the present invention. When the excipient is lactose it will typically be present as milled lactose, wherein not more than 85% of lactose particles will have a MMD of 60-90 μm and not less than 15% will have a MMD of less than 15 μm. Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulization may be formulated with an aqueous vehicle with the addition of agents such as acid or alkali, buffer salts, isotonicity adjusting agents or antimicrobials. They may be sterilized by filtration or heating in an autoclave, or presented as a non-sterile product. Suitably, administration by inhalation may preferably target the organ of interest for respiratory diseases, i.e. the lung, and in doing so may reduce the efficacious dose needed to be delivered to the patient. In addition, administration by inhalation may reduce the systemic exposure of the compound thus avoiding effects of the compound outside the lung.

Recently, pharmaceutical compositions have been developed for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

### REFERENCES

1) Morohashi, Mukaida. J. Leukocyte Biol, 1995. 57:180.
2) McColl, Clark-Lewis. J. Immunology, 1999, 163:2829.
3) Jones, Everard. Eur Respir J 2002; 20: 651-657.
4) Everard, Milner. Archives of Disease in Childhood. 1994; 71: 428-432.
5) Smith, Forsyth.. 2001, J. Paediatr. Child Health 37:146.
6) McNamara, Smyth. The Journal of Infectious Diseases 2005; 191:1225-32.
7) Hull, Kwiatkowski. Thorax 2000;55:1023-1027.
8) Goetghebuer, Hull. Clin Exp Allergy 2004; 34:801-803.
9) Waering. Sarawar. Viral Immunology, Volume 20, Number 3, 2007.
10) Tate, Reading. Plos One. March 2011, Volume 6, Issue 3.
11) Miller, Lukacs. The Journal of Immunology, 2003, 170: 3348-3356.
12) Sakai, Ochiai. Journal of Virology. Mar. 2000, p. 2472-2476.

1. A composition comprising danirixin in combination with a neuraminidase inhibitor compound.

2. A composition comprising danirixin in combination with zanamivir.

3. A composition comprising danirixin in combination with oseltamivir.

4.-5. (canceled)

6. A composition comprising danirixin in combination with one or more antimicrobial agents selected from Table 4.

7. A pharmaceutical composition comprising danirixin in combination with a neuraminidase inhibitor compound and a pharmaceutically acceptable excipient.

8. A pharmaceutical composition comprising danirixin in combination with zanamivir and a pharmaceutically acceptable excipient.

9. A pharmaceutical composition comprising danirixin in combination with oseltamivir and a pharmaceutically acceptable excipient.

10. (canceled)

11. A pharmaceutical composition comprising danirixin in combination with one or more antimicrobial agents selected from Table 4 and a pharmaceutically acceptable excipient.

12. A method for treating a respiratory infectious disease in a subject, the method comprising administering to a subject suffering from a respiratory infectious disease danirixin in combination with a neuraminidase inhibitor compound.

13. A method for treating a respiratory infectious disease in a subject, the method comprising administering to a subject suffering from a respiratory infectious disease danirixin in combination with zanamivir.

14. A method for treating a respiratory infectious disease in a subject, the method comprising administering to a subject suffering from a respiratory infectious disease danirixin in combination with oseltamivir.

15.-16. (canceled)

17. A method for treating a respiratory infectious disease in a subject, the method comprising administering to a subject suffering from a respiratory infectious disease danirixin in combination with one or more antimicrobial agents selected from Table 4.

18. The method according to any one of claims 12-17, wherein the respiratory infectious disease is influenza.

19. The method according to any one of claims 12-17, wherein the respiratory infectious disease is RSV.

20. The method according to any one of claims 12-17, wherein the combination of danirixin and neuraminidase inhibitor compound are administered in the same dosage form.

21. The method according to any one of claims 12-17, wherein the combination of danirixin and neuraminidase inhibitor compound are administered simultaneously.

22. The method according to any one of claims 12-17, wherein the combination of danirixin and neuraminidase inhibitor compound are administered separately.

23. The method according to any one of claims 12-17, wherein the combination of danirixin and neuraminidase inhibitor compound are administered in the same dosage form.

24. The method according to any one of claims 12-17, wherein the combination of the compound of danirixin and neuraminidase inhibitor compound are administered simultaneously.

25. The method according to any one of claims 12-17, wherein the combination of danirixin and neuraminidase inhibitor compound are administered separately.

26.-28. (canceled)

29. A method for treating influenza in a subject, the method comprising administering danirixin to a subject suffering from influenza.

30. A method for treating RSV in a subject, the method comprising administering danirixin to a subject suffering from RSV.

31. A pharmaceutical composition for intravenous administration comprising: danirixin as a hydrobromide salt in aqueous solution.

32. A pharmaceutical composition for intravenous administration comprising: danirixin as a hydrobromide salt and a pharmaceutically acceptable excipient in aqueous solution.

33.-35. (canceled)

36. The pharmaceutical composition according to claim 32, wherein the pharmaceutically acceptable excipient comprises Captisol®.

37. A method for treating a respiratory infectious disease in a subject, the method comprising administering the pharmaceutical composition according to any one of claims 31-36, to a subject suffering from a respiratory infectious disease.

38. A method for treating RSV in a subject, the method comprising administering the pharmaceutical composition according to any one of claims 31-36, to a subject suffering from RSV.

39. A method for treating influenza in a subject, the method comprising administering the pharmaceutical composition according to any one of claims 31-36, to a subject suffering from influenza.

40.-42. (canceled)
